(19) United States
(12) Patent Application Publication
(10) Pub. No.: US 2024/0261575 A1
(43) Pub. Date: Aug. 8, 2024

(54) HEART SOUND BASED BUNDLE BRANCH BLOCK DETECTION AND PACING OPTIMIZATION

(71) Applicant: Cardiac Pacemakers, Inc., St. Paul, MN (US)

(72) Inventors: Mojgan Goftari, Shoreview, MN (US); Jonathan Bennett Shute, Eagan, MN (US); Viktoria A. Averina, Shoreview, MN (US); Pramodsingh Hirasingh Thakur, Woodbury, MN (US)

(21) Appl. No.: 18/433,826

(22) Filed: Feb. 6, 2024

Related U.S. Application Data
(60) Provisional application No. 63/443,833, filed on Feb. 7, 2023.

(51) Int. Cl. A61N 1/365; A61N 1/05; A61N 1/368; A61N 1/372; G16H 50/30

(52) U.S. Cl. CPC A61N 1/36578; A61N 1/0587; A61N 1/36585; A61N 1/36843; A61N 1/3688; A61N 1/37247; G16H 50/30

(57) ABSTRACT

Systems and methods for detecting a blockage in a patient's cardiac conduction system using heart sound information is disclosed. An exemplary medical-device system includes a data receiver circuit to receive heart sound information sensed from a patient, and a controller circuit to generate a heart sound metric or characteristic from the received heart sound information. The heart sound metric can include one indicative of a presence or absence of split S1 sound. The controller circuit can detect a bundle branch block (BBB), including to discriminate a left bundle branch block (LBBB) from a right bundle branch block (RBBB), based at least in part on the heart sound metric. The BBB indicator can be provided to a user or a process executable by the medical-device system to optimize cardiac pacing and restore cardiac synchrony.

### CLAIM OF PRIORITY

This application claims the benefit of U.S. Provisional Application No. 63/443,833 filed on Feb. 7, 2023, which is hereby incorporated by reference in its entirety.

### TECHNICAL FIELD

This document relates generally to medical systems, and more particularly, to systems, devices and methods for detecting a blockage in a patient's cardiac conduction system based on heart sounds.

### BACKGROUND

The heart is the center of a person's circulatory system. It includes an electro-mechanical system performing two major pumping functions. The left portions of the heart, including the left atrium (LA) and left ventricle (LV), draw oxygenated blood from the lungs and pump it to the organs of the body to provide the organs with their metabolic needs for oxygen. The right portions of the heart, including the right atrium (RA) and right ventricle (RV), draw deoxygenated blood from the body organs and pump it to the lungs where the blood gets oxygenated. These pumping functions result from contractions of the myocardium (cardiac muscles). In a normal heart, the sinoatrial (SA) node, the heart's natural pacemaker, generates electrical pulses, called action potentials, which propagate through natural electrical conduction pathways known as His-Purkinje system to various regions of the heart to excite the myocardial tissue of the heart. For example, the action potentials originated from the SA node propagate through the atrioventricular (AV) node, the His bundle (also known as Bundle of His), the bundle branches, and Purkinje fibers to reach the ventricular myocardium, resulting in coordinated contractions in both ventricles.

Coordinated delays in the propagation of the action potentials in a normal cardiac electrical conduction system cause the various portions of the heart to contract in synchrony to result in efficient pumping functions. A blockage or disruption in the cardiac conduction system and/or deteriorated myocardium may cause dyssynchronous contraction of the heart, resulting in poor hemodynamic performance, including a diminished blood supply to the heart and the rest of the body. For example, an abnormal delay in the transmission of the action potentials in the His bundle may cause irregular or dyssynchronous contractions of the ventricles. A blockage or disruption in any part of the bundle branch may lead to a bundle branch block, such as left bundle branch block (LBBB) or right bundle branch block (RBBB). While RBBB is generally more common in general population, approximately one third of the heart failure patients have LBBB. LBBB may also be used as an indicator of acute myocardial infarction (MI) which could lead to an ischemic stroke.

Artificial cardiac pacing system have been used to rectify cardiac dyssynchrony and to improve hemodynamic performance. The artificial cardiac pacing system may provide electrical stimulations to one or more portions of the heart such as to restore normal functioning of the heart to a certain extent. For example, right ventricular (RV) pacing via electrodes implanted in the apex of the RV have been used in both single ventricular and biventricular (BiV) pacing. RV apical pacing directly excites the ventricular myocardium in an unnatural way involving slow muscle-muscle contraction. Normally, ventricular excitation involves rapid coordinate activation via action potentials that are propagated through the natural conduction pathways. Studies have shown that, in some patients, the cardiac dyssynchrony induced by RV apical pacing, partially due to the interventricular delay in impulse propagation to the left ventricle, can result in adverse ventricular changes including enlarged ventricles and heart failure when delivered over the long term. Moreover, permanent changes in myocardial perfusion and structure may develop over time in these patients, which may further decrease cardiac output and deteriorate ventricular function. BiV pacing involves RV pacing via one lead, and LV pacing via another lead, and has been demonstrated to restore synchronized contraction of both ventricles. However, the potential adverse impact on ventricular function produced by the RV apical pacing may still exist in BiV pacing. Muscle-muscle activation via epicardial LV pacing is also known to result in a less vigorous contraction.

Heart sounds are generally associated with mechanical vibration of a heart and blood flow through the heart. Heart sounds recur with each cardiac cycle and are separated and classified according to the activity associated with the vibration. Historically, heart sounds were assessed by humans and thus only audible portion of the vibration was used. Devices can now assess full spectrum of cardiac vibrations which include both audible and subaudible components, thus the term “sound” in this document refers to the full spectrum of vibrations. Typically, heart sounds sensed from a subject may include several components within a cardiac cycle, including a first (S1), a second (S2), a third (S3), or a fourth (S4) heart sound. S1 is associated with the vibrations produced by the heart during closure of the atrioventricular (mitral and tricuspid) valves as the ventricular pressures exceed atrial pressures at the beginning of systole. S2 is produced by closure of the aortic and pulmonary valves, and marks the beginning of diastole. S3 is produced by early diastolic vibrations corresponding to passive ventricular filling during diastole, when the blood rushes into the ventricles. S4 is produced by late diastolic vibrations corresponding to active ventricular filling when the atria contract and push the blood into the ventricles. In a healthy subject, S3 is usually faint and S4 is rarely audible. However, a pathologic S3 or S4 may be higher pitched and louder.

Heart sounds have been used to assess cardiac systolic and diastolic functions. Systole is the contraction or a period of contraction of the heart that causes blood to be forced out of the heart such as the ventricles and into the aorta and pulmonary artery. Diastole is the relaxation or a period of relaxation of the heart during which the blood flows back into the heart such as the ventricles. Patients with cardiac diseases may have deteriorated systolic or diastolic functions. For example, congestive heart failure (CHF) occurs when the heart is unable to supply enough blood to maintain a healthy physiologic state.

### OVERVIEW

Hemodynamic response to cardiac pacing may depend on many factors, including pacing site selection and pacing configurations. Many patients receiving cardiac pacing therapy have intact cardiac conduction system (e.g., the His-Purkinje system) in the ventricles, and therefore having normal ventricular activation. Conventional cardiac pacing such as long-term RV apical pacing (i.e., pacing at RV apex) may cause a decrease in cardiac output and efficiency due to the uncoordinated contraction sequence. This dyssynchrony may eventually cause adverse long-term effects. Dyssynchronous contraction of the ventricles occurs during conventional RV pacing because the activation sequence may be much slower and propagate slowly from the right to the left ventricle across the interventricular septum, thereby causing ventricular dyssynchrony. This sequence of activation results in an uncoordinated contraction, which does not occur during biventricular activation through the natural conduction system of the heart.

Pacing a portion of the natural cardiac conduction system (e.g., the His bundle, left or right bundle branches, fascicles, or Purkinje fibers, among other conductive cardiac tissue) is an alternative to conventional ventricular pacing. The cells of the natural conduction system may propagate an activation signal about four times faster than working myocardium. Conduction system pacing (CSP), such as His bundle pacing (HBP), septal pacing such as left bundle branch pacing (LBBP) or right bundle branch pacing (RBBP) at a septal region, or pacing at other parts the His-Purkinje conduction system, may activate the heart's natural electrical conduction system, restore or improve cardiac synchrony, reduce or even eliminate potential long-term harmful hemodynamic effects associated with conventional RV apical pacing.

However, when not being successful, the CSP may not adequately restore cardiac synchrony. For example, HBP may lose its ability to activate (or capture) the His bundle or neighboring conductive tissue, but only activates the para-Hisian myocardium. Stimulating muscles near the His bundle may cause dyssynchronous patterns similar to RV apical pacing. This undesirable outcome is referred to as para-Hisian capture. Simultaneous capture of the His bundle and para-Hisian muscle (also known as a non-selective His bundle capture) may be as clinically effective as the His bundle only capture (also known as a selective His bundle capture, as the resultant ventricular activation and contraction is dominated by the more rapid conducting Purkinje system. Another undesirable outcome of HBP is known as a complete loss of capture (LOC), where the pacing pulses capture neither the para-Hisian myocardium nor the His bundle.

The ability of CSP to restore cardiac synchrony may also be dependent on the pacing site relative to the blockage site along the His-Purkinje system, such as blockage of the His bundle or a bundle branch. Ventricular dyssynchrony in many CHF patients may be attributed to various degrees of left bundle branch block (LBBB), which causes delayed LV depolarization lagging behind RV depolarization. Right bundle branch block (RBBB) is generally more common than LBBB in general population. Effective propagation of the action potentials through the His-Purkinje system to restore cardiac synchrony may be achieved only if the pacing pulses are delivered distal to the blockage site. If the pacing pulses are delivered proximal to the blockage site, even if the proximal portion of the His bundle is activated, the action potential cannot bypass the blockage and propagate to the ventricles through the His-Purkinje system. Consequently, no cardiac synchrony may be restored.

Proper detection of a blockage along the cardiac conduction system, such as detection and discrimination of LBBB from RBBB, can provide a guidance to optimize cardiac pacing. Pacing site is conventionally determined by analyzing cardiac electrical signals such as surface electrocardiogram (ECG). ECG parameters such as QRS width has been used to quantify a degree of dyssynchrony and to identify an “optimal” or a desired pacing site at which the pacing would result in a shortening of QRS width. However, complex computation is generally required to have a robust and accurate QRS width measurement, which can be timing consuming. Stability of QRS width measurement may vary significantly in some post-implant patients. Additionally, while QRS width or other ECG parameters are indicative of or correlated to electrical dyssynchrony (or a restoration to synchrony), the QRS width and the ECG parameters may not adequately indicate or correlate to mechanical dyssynchrony between left and right ventricles or various portions of a ventricle. The present inventors have recognized a technical challenge in cardiac conduction system pacing, particularly more effective and efficient techniques to determine pacing locations at or near the cardiac conduction system to reliably capture the conductive tissue and restore ventricle synchrony.

The present document describes techniques to detect a blockage in the cardiac conduction system, particularly to distinguish between LBBB and RBBB, using heart sound information. Information about the detected blockage can be used to optimize cardiac pacing therapy, such as selecting a proper CSP pacing site or programming pacing parameters. An exemplary medical-device system includes a data receiver circuit to receive heart sound information sensed from a patient, and a controller circuit to generate a heart sound metric or characteristic from the received heart sound information. The controller circuit can detect a bundle branch block (BBB), including to discriminate a left bundle branch block (LBBB) from a right bundle branch block (RBBB), based at least in part on the heart sound metric. The BBB indicator can be provided to a user (e.g., clinician) or a process executable by the medical-device system to optimize cardiac pacing to restore cardiac synchrony or to improve cardiac performance.

Example 1 is a medical-device system for managing a heart condition, comprising: a data receiver circuit configured to receive heart sound information sensed from a patient, the heart sound information including an S1 sound signal; and a controller circuit configured to: generate a heart sound metric using the received heart sound information; detect bundle branch block (BBB), including to discriminate a left bundle branch block (LBBB) from a right bundle branch block (RBBB), based at least in part on the generated heart sound metric; and provide information about the detected BBB to a user or a process executable by the medical-device system.

In Example 2, the subject matter of Example 1 optionally includes the heart sound metric that is indicative of a presence or absence of split S1 sound, wherein to detect the BBB, the controller circuit is configured to: detect the LBBB in response to an absence of the split S1 sound or an occurrence rate of the split S1 sound falling below a threshold; and detect the RBBB in response to a presence of the split S1 sound or the occurrence rate of the split S1 exceeding the threshold.

In Example 3, the subject matter of Example 2 optionally includes the heart sound metric indicative of the presence or absence of split S1 sound which can include a signal width of the S1 sound signal, wherein the controller circuit is configured to detect the presence or absence of split S1 sound based on a comparison of the signal width of the S1 sound signal to a width threshold.

In Example 4, the subject matter of any one or more of Examples 2-3 optionally includes the heart sound metric indicative of the presence or absence of split S1 sound, which can include a spectral entropy of the S1 sound signal, wherein the controller circuit is configured to detect the presence or absence of split S1 sound based on a comparison of the spectral entropy of the S1 sound signal to a spectral entropy threshold.

In Example 5, the subject matter of any one or more of Examples 2-4 optionally includes an electrostimulator configured to provide cardiac stimulation to the patient, wherein the controller circuit is configured to generate a control signal to the electrostimulator to: in response to the detected LBBB, deliver cardiac conduction system pacing (CSP) at a His bundle or septal region of the heart via first one or more electrodes; and in response to the detected RBBB, withhold the cardiac stimulation, or deliver right-ventricular (RV) pacing at an RV apical site of the heart via second one or more electrodes.

In Example 6, the subject matter of Example 5 optionally includes a lead having a distal portion configured to be inserted into the His bundle or septal region at adjustable depth to deliver the CSP therein via the first one or more electrodes associated with the lead, wherein, in response to the detection of LBBB, the controller circuit is configured to: receive paced heart sound information in response to the delivery of the CSP; generate a heart sound metric indicative of a presence or absence of split S1 sound using the paced heart sound information; and determine whether or not to adjust a depth of insertion of the distal portion of the lead into the His bundle or septal region, including not to adjust the depth of insertion if the heart sound metric indicates a presence of split S1 sound, or if an occurrence rate of the split S1 sound exceeds a rate threshold, and to adjust the depth of insertion if the heart sound metric indicates an absence of split S1 sound, or if the occurrence rate of the split S1 sound falls below the rate threshold.

In Example 7, the subject matter of Example 6 optionally includes the controller circuit that can be configured to generate a control signal to a user interface to display in real time the heart sound metric indicative of the presence or absence of split S1 sound while the CSP is being delivered with the distal portion of the lead being positioned at varying depths of insertion.

In Example 8, the subject matter of any one or more of Examples 5-7 optionally includes the controller circuit that can be configured to, in response to the detection of LBBB: generate a control signal to the electrostimulator to deliver cardiac resynchronization pacing (CRT) to left and right ventricles of the heart in accordance with a CRT pacing parameter; receive paced heart sound information in response to the delivery of the CRT; generate a heart sound metric indicative of a presence or absence of split S1 sound using the paced heart sound information; and determine whether or not to adjust the CRT pacing parameter, including not to adjust the CRT pacing parameter if the heart sound metric indicates a presence of split S1 sound, or if an occurrence rate of the split S1 sound exceeds a rate threshold, and to adjust the CRT pacing parameter if the heart sound metric indicates an absence of split S1 sound, or if the occurrence rate of the split S1 sound falls below the rate threshold.

In Example 9, the subject matter of Example 8 optionally includes, wherein to adjust the CRT pacing parameter includes to adjust a pacing dosage parameter or a pacing timing parameter, or to switch from a single-site ventricular pacing to multi-site ventricular pacing.

In Example 10, the subject matter of any one or more of Examples 8-9 optionally includes the controller circuit that can be configured to generate a control signal to a user interface to display in real time the heart sound metric indicative of the presence or absence of split S1 sound while the CRT is being delivered in accordance with varying values of the CRT pacing parameter.

In Example 11, the subject matter of any one or more of Examples 1-10 optionally includes the data receiver circuit that can be configured to receive respiration information sensed from the patient, the respiration information including an inspiration phase and an expiration phase in a respiration cycle, wherein the controller circuit is configured to generate the heart sound metric using a portion of the received heart sound information corresponding to the inspiration phase of the respiration cycle, and to detect the BBB based on the heart sound metric during the inspiration phase.

In Example 12, the subject matter of any one or more of Examples 1-11 optionally includes the heart sound metric that can include an intensity of the S1 sound signal, wherein the controller circuit is configured to detect the BBB further based on the S1 sound intensity.

In Example 13, the subject matter of any one or more of Examples 1-12 optionally includes the data receiver circuit that can be configured to receive cardiac electrical activity information sensed from the patient, wherein the controller circuit is configured to generate the heart sound metric including a measurement of pre-ejection period using the received heart sound information and the cardiac electrical activity information, and to detect the BBB further based on the measurement of pre-ejection period.

In Example 14, the subject matter of any one or more of Examples 1-13 optionally includes a risk assessment circuit configured to generate a risk of cardiac or pulmonary disease in the patient based at least on the information of the detected BBB.

In Example 15, the subject matter of any one or more of Examples 1-14 optionally includes a user interface configured to generate an alert of the detected BBB.

Example 16 is a method of managing a heart condition using a medical-device system, the method comprising: receiving heart sound information sensed from a patient, the heart sound information including an S1 sound signal; generating a heart sound metric using the received heart sound information; detecting bundle branch block (BBB), including discriminating a left bundle branch block (LBBB) from a right bundle branch block (RBBB) based at least in part on the generated heart sound metric; and providing information about the detected BBB to a user or a process.

In Example 17, the subject matter of Example 16 optionally includes the heart sound metric that can be indicative of a presence or absence of split S1 sound, wherein detecting the BBB includes detecting the LBBB in response to an absence of the split S1 sound or an occurrence rate of the split S1 sound falling below a threshold, and detecting the RBBB in response to a presence of the split S1 sound or the occurrence rate of the split S1 exceeding the threshold.

In Example 18, the subject matter of Example 17 optionally includes the heart sound metric indicative of the presence or absence of split S1 sound, which can include a signal width of the S1 sound signal, the method comprising detecting the presence or absence of split S1 sound based on a comparison of the signal width of the S1 sound signal to a width threshold.

In Example 19, the subject matter of any one or more of Examples 17-18 optionally includes the heart sound metric indicative of the presence or absence of split S1 sound, which can include a spectral entropy of the S1 sound signal, the method comprising detecting the presence or absence of split S1 sound based on a comparison of the spectral entropy of the S1 sound signal to a spectral entropy threshold.

In Example 20, the subject matter of any one or more of Examples 17-19 optionally include: in response to the detected LBBB, delivering cardiac conduction system pacing (CSP) at a His bundle or septal region of the heart via first one or more electrodes; and in response to the detected RBBB, withholding cardiac stimulation, or delivering right-ventricular (RV) pacing at an RV apical site of the heart via second one or more electrodes.

In Example 21, the subject matter of Example 20 optionally includes, in response to the detection of LBBB: receiving paced heart sound information in response to the delivery of the CSP at the His bundle or septal region of the heart; generating a heart sound metric indicative of a presence or absence of split S1 sound using the paced heart sound information; and determining whether or not to adjust a depth of insertion of a distal portion of a lead into the His bundle or septal region to deliver the CSP therein via the first one or more electrodes associated with the lead, including not adjusting the depth of insertion if the heart sound metric indicates a presence of split S1 sound, or if an occurrence rate of the split S1 sound exceeds a rate threshold, and adjusting the depth of insertion if the heart sound metric indicates an absence of split S1 sound, or if the occurrence rate of the split S1 sound falls below the rate threshold.

In Example 22, the subject matter of any one or more of Examples 20-21 optionally includes, in response to the detection of LBBB: delivering cardiac resynchronization pacing (CRT) to left and right ventricles of the heart in accordance with a CRT pacing parameter; receiving paced heart sound information in response to the delivery of the CRT; generating a heart sound metric indicative of a presence or absence of split S1 sound using the paced heart sound information; and determining whether or not to adjust the CRT pacing parameter, including not adjusting the CRT pacing parameter if the heart sound metric indicates a presence of split S1 sound, or if an occurrence rate of the split S1 sound exceeds a rate threshold, and adjusting the CRT pacing parameter if the heart sound metric indicates an absence of split S1 sound, or if the occurrence rate of the split S1 sound falls below the rate threshold.

In Example 23, the subject matter of any one or more of Examples 16-22 optionally includes receiving respiration information sensed from the patient, the respiration information including an inspiration phase and an expiration phase in a respiration cycle, wherein detecting the BBB is based at least in part on the heart sound metric that is generated using a portion of the received heart sound information corresponding to the inspiration phase of the respiration cycle.

The systems, devices, and methods discussed in this document improve the technology of device-based monitoring and management of cardiac conditions such as BBB, yet with little to no additional cost or system complexity. The heart sound-based techniques for detecting a blockage or disruption in the cardiac conduction system, particularly to discriminate LBBB from RBBB, can help determine proper pacing locations and electrode configurations for cardiac pacing (e.g., CSP) to restore cardiac synchrony or to improve cardiac performance in patients with heart failure. CSP may activate the His-Purkinje system, thereby preserving ventricular synchrony and improving cardiac performance without structural and functional impairment to the heart. Compared to conventional ECG-based pacing optimization techniques, the heart sound-based pacing optimization as described in the present document takes advantage of existing sensors (e.g., accelerometers) capable of measuring heart sounds. The heart sound metrics can be derived from the heart sound signal using less complicated techniques that require less computation time. The heart sound-based pacing site and pacing parameter optimization can increase the chance of capturing the cardiac conductive system and thus improve the overall success rate of CSP therapy, promote synchrony via patient's native His-Purkinje system, and produce favorable cardiac performance and hemodynamic outcome. With more effective and efficient pacing site optimization as described in this document, ineffective pacing (i.e., loss of capture) can be reduced, and battery power of an cardiac pacing system or device can be saved, and extend device longevity can be achieved without compromising effective CSP therapy. As such, the subject matter discussed in this document improves the functionality of an cardiac pacing system or device.

In addition to optimizing cardiac pacing such as CSP, the heart sound-based BBB detection and discrimination between LBBB and RBBB as described in present document in accordance with various embodiments may also be used in detection, risk stratification, and therapy titration for other cardiac or pulmonary conditions. For example, RBBB can be resulted from pulmonary hypertension, LBBB is a risk factor for coronary artery disease such as acute myocardial infarction (MI). The heart sound-based detection of RBBB as described herein can be used to predict a risk of, and thus help prevent, pulmonary hypertension. The heart sound-based detection of LBBB as described herein can be used to predict a risk of, and thus help prevent, acute MI and ischemic stroke.

While His bundle pacing or bundle branch pacing (e.g., LBB pacing or RBB pacing) are specifically discussed in various examples in this document, this is meant only by way of example and not limitation. It is within the contemplation of the inventors, and within the scope of this document, that the systems, devices, and methods described in accordance with various embodiments herein may be similarly applied to controllably stimulate other locations at or near the cardiac conduction system including, for example, His bundle, fascicles, Purkinje fibers, among other conductive tissue of the cardiac conduction system, and to determine an optimal or desired pacing location and optimal or desired pacing parameters.

This Overview provides some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIG. 2 illustrates generally an example of a cardiac disease management system and portions of an environment in which the system may operate.

FIG. 3 illustrates generally an example of a heart sound-based cardiac monitor and pacing system.

FIGS. 4A-4C illustrates conceptually different patterns of split S1 sound in normal heart (with intact cardiac conduction system), RBBB, and LBBB.

FIGS. 5A-5B illustrate examples of changes in S1 morphology and S1 intensity before and after myocardial infarction in associated with LBBB in a patient.

FIG. 6 illustrates generally an example method for detecting an managing blockage in a patient's cardiac conduction system such as BBB.

FIGS. 7A-7C illustrate generally example methods for optimizing cardiac pacing therapy using heart sound-based BBB detection to restore cardiac synchrony.

FIG. 8 illustrates generally a block diagram of an example machine upon which any one or more of the techniques (e.g., methodologies) discussed herein may perform.

### DETAILED DESCRIPTION

Disclosed herein are systems, devices, and methods for detecting a blockage in a patient's cardiac conduction system using heart sound information sensed from a patient. An exemplary medical-device system includes a data receiver circuit to receive heart sound information sensed from a patient, and a controller circuit to generate a heart sound metric or characteristic from the received heart sound information. The controller circuit can detect a bundle branch block (BBB), including to discriminate a left bundle branch block (LBBB) from a right bundle branch block (RBBB), based at least in part on the heart sound metric. The BBB indicator can be provided to a user (e.g., clinician) or a process executable by the medical-device system to optimize cardiac pacing to restore cardiac synchrony or to improve cardiac performance.

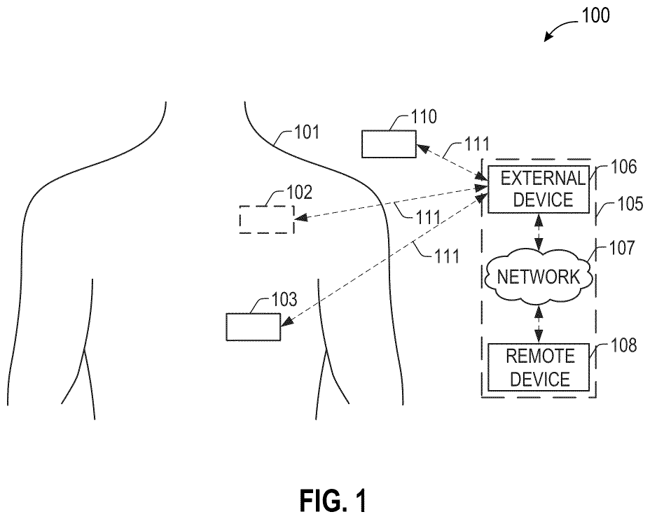

FIG. 1 illustrates generally an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 may include one or more ambulatory medical devices, an external system 105, and a communication link 111 providing for communication between the one or more ambulatory medical devices and the external system 105. The one or more ambulatory medical devices may include an implantable medical device (IMD) 102, a wearable medical device (WMD) 103, or one or more other implantable, leadless, subcutaneous, external, wearable, or ambulatory medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 may include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 may include a monitor implanted, for example, subcutaneously in the chest of patient 101, the IMD 102 including a housing containing circuitry and, in certain examples, one or more sensors, such as a temperature sensor, etc.

The IMD 102 may include an assessment circuit configured to detect or determine specific physiologic information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy may include delivery of one or more drugs to the patient 101, such as using the IMD 102 or one or more of the other ambulatory medical devices, etc. In some examples, therapy may include cardiac resynchronization therapy for rectifying dyssynchrony and improving cardiac function in heart failure patients. In other examples, the IMD 102 may include a drug delivery system, such as a drug infusion pump to deliver drugs to the patient for managing arrhythmias or complications from arrhythmias, hypertension, or one or more other physiologic conditions. In other examples, the IMD 102 may include one or more electrodes configured to stimulate the nervous system of the patient or to provide stimulation to the muscles of the patient airway, etc.

The WMD 103 may include one or more wearable or external medical sensors or devices (e.g., automatic external defibrillators (AEDs), Holter monitors, patch-based devices, smart watches, smart accessories, wrist-or finger-worn medical devices, such as a finger-based photoplethysmography sensor, etc.).

In an example, the IMD 102 or the WMD 103 may include or be coupled to an implantable or wearable sensor to sense a heart sound signal, and include a heart sound recognition circuit to recognize one or more heart sound components such as S1, S2, S3, or S4. Also included in the IMD 102 or the WMD 103 is a heart sound-based event detector circuit that can detect a physiologic event (e.g., a cardiac arrhythmia episode, or a worsening heart failure (WHF) event) based at least on a heart sound metric of the detected one or more heart sound component. Examples of such heart sound metric may include an amplitude, or timing of the heart sound component within a cardiac cycle relative to a fiducial point. In some examples, at least a portion of the heart sound recognition circuit and/or the heart sound-based event detector circuit may be implemented in and executed by the external system 105.

The external system 105 may include a dedicated hardware/software system, such as a programmer, a remote server-based patient management system, or alternatively a system defined predominantly by software running on a standard personal computer. The external system 105 can manage the patient 101 through the IMD 102 or one or more other ambulatory medical devices connected to the external system 105 via a communication link 111. In other examples, the IMD 102 can be connected to the WMD 103, or the WMD 103 can be connected to the external system 105, via the communication link 111. This may include, for example, programming the IMD 102 to perform one or more of acquiring physiological data, performing at least one self-diagnostic test (such as for a device operational status), analyzing the physiological data, or optionally delivering or adjusting a therapy for the patient 101. Additionally, the external system 105 can send information to, or receive information from, the IMD 102 or the WMD 103 via the communication link 111. Examples of the information may include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 may include an external device 106 in proximity of the one or more ambulatory medical devices, and a remote device 108 in a location relatively distant from the one or more ambulatory medical devices, in communication with the external device 106 via a communication network 107. Examples of the external device 106 may include a medical device programmer. The remote device 108 can be configured to evaluate collected patient or patient information and provide alert notifications, among other possible functions. In an example, the remote device 108 may include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server may include a memory device to store the data in a patient database. The server may include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications may include a Web page update, phone or pager call, E-mail, SMS, text or “Instant” message, as well as a message to the patient and a simultaneous direct notification to emergency services and to the clinician. Other alert notifications are possible. The server may include an alert prioritizer circuit configured to prioritize the alert notifications. For example, an alert of a detected physiologic event can be prioritized using a similarity metric between the physiological data associated with the detected physiologic event to physiological data associated with the historical alerts.

The remote device 108 may additionally include one or more locally configured clients or remote clients securely connected over the communication network 107 to the server. Examples of the clients may include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can output the detected physiologic events to a system user, such as the patient or a clinician, or to a process including, for example, an instance of a computer program executable in a microprocessor. In an example, the process may include an automated generation of recommendations for anti-arrhythmic therapy, or a recommendation for further diagnostic test or treatment. In an example, the external device 106 or the remote device 108 may include a respective display unit for displaying the physiologic or functional signals, or alerts, alarms, emergency calls, or other forms of warnings to signal the detection of arrhythmias. In some examples, the external system 105 may include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” may include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 may include a local external implantable medical device programmer. The external system 105 may include a remote patient management system that can monitor patient status or adjust one or more therapies such as from a remote location.

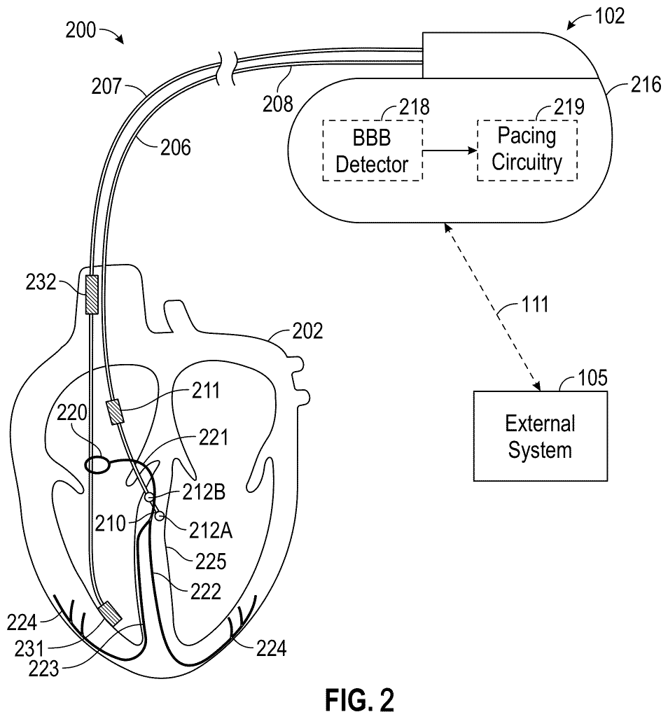

FIG. 2 is a schematic diagram illustrating an embodiment of a cardiac disease management system 200 and portions of an environment in which the system 200 may operate. The cardiac disease management system 200 may perform a range of activities, including remote patient monitoring, diagnosis of a disease condition, and providing a therapy to treat the disease condition and to improve patient outcome. In an example, the therapy may include conduction system pacing (CSP), such as His bundle pacing (HBP), septal pacing such as left bundle branch pacing (LBBP) or right bundle branch pacing (RBBP), or pacing at other parts the His-Purkinje conduction system. One or more of these activities may be performed proximal to a patient (e.g., in the patient's home or office), through a centralized server (e.g., in a hospital, clinic or physician's office), or through a remote workstation (e.g., a secure mobile computing device).

The cardiac disease management system 200 may be coupled to a patient's heart 202. The cardiac disease management system 200 includes an ambulatory medical device (AMD), which can be an implantable device subcutaneously implanted in a chest, abdomen, or other parts of the patient, a subcutaneous monitor or diagnostic device, or a wearable medical device such as a patch-based device or a smart wearable or accessory, among others. In the example as illustrated in FIG.2 , the AMD can be an implantable medical device, such as the IMD 102 as illustrated in FIG. 1.

The cardiac disease management system 200 may include a lead system electrically coupled to the IMD 102. The lead system may include one or more transvenously, percutaneously, or minimally invasive placed leads or catheters. Each lead or catheter may include one or more electrodes. The arrangements and uses of the lead system and the associated electrodes may be determined by patient need and capability of the IMD 102. The associated electrodes on the lead system may be positioned at the patient's thorax or abdomen to sense a physiological signal indicative of cardiac activity, or a physiological response to stimulation of a target tissue. The lead system may be surgically inserted into, or positioned on the surface of, a heart 202. The electrodes associated with the lead system may be disposed in a target site in a right atrium (RA), a right ventricle (RV), a left atrium (LA), or a left ventricle (LV), or other body parts. Stimulation energy may be delivered to a target site via one or more of these electrodes. Some electrodes may be used for sensing cardiac activity, such as an intrinsic or evoked cardiac electrical activity.

In the illustrated example, the lead system may include a lead 206 having a proximal end 208 configured to be connected to the IMD 102, and a distal end 210 that includes one or more electrodes configured to deliver stimulation energy, such as in a form of pacing pulses, to the His bundle 221. FIG.2 illustrates, by way of example and not limitation, two electrodes including a tip electrode 212A and a ring electrode 212B. Additional electrodes may be included in the lead 206 for sensing electrical activity or for delivering stimulation energy, such as an atrial lead 211. The lead 206 may be placed such that one or more electrodes, such as electrodes 212A-212B, are positioned in or near a His bundle 221, a region distal to the blocked or slowly conducting AV node, an interventricular septum region, or a right atrial region near the His bundle 221. Alternatively, one or more of the electrodes 212A-212B, or other electrodes on the lead 206, can be positioned at the interventricular septum 225 to provide septal pacing, which may include one or more of left bundle branch (LBB) pacing or right bundle branch (RBB) pacing. As part of the natural electrical conduction system of the heart 202, the His bundle 221 transmits the electrical impulses from the AV node 220 to the point of the apex of the fascicular branches via the left bundle branch 222 and the right bundle branch 223 within the interventricular septum 225. The left bundle branch 222 and the right bundle branch 223 leads to the Purkinje fibers 224, which provide electrical conduction to the ventricles, causing the ventricles to contract. In some examples, the lead 206 may be placed such that one or more electrodes associated with the lead 206, such as electrodes 212A-212B, are positioned at or near other parts of the natural cardiac conduction system, such as one of the bundle branches 222 or 223, the Purkinje fibers 224, or other conductive tissue, in addition to or in lieu of a region at or near the His bundle 221. In some example, the distal tip of the lead 206 may be inserted into the His bundle or a septal region (proximal to a portion of LBBB or a portion of RBBB therein) at a desired depth, such that one or more of the electrodes 212A-212B are in closer proximity to the cardiac conductive tissue of the His-Purkinje system, thereby increasing the success rate of capturing the cardiac conductive tissue via electrical stimulation.

In some examples, one or more leads other than the lead 206 may additionally or alternatively be used to provide CSP (e.g., HBP, septal pacing such as LBBP or RBBP, among others) via one or more electrodes associated therewith. Such leads may include transvenous, subcutaneous, endocardial, epicardial, or non-invasive leads. For example, a ventricular lead 207 may include electrodes configured to be positioned at the septum 225, or optionally inserted into a septal region at a desired depth, to provide septal pacing such as LBBP or RBBP therein. The ventricular lead 207 may additionally include one or more electrodes, such as a distal electrode 231 and a proximal electrode 232, for cardiac sensing and/or pacing the ventricle.

In an example, the lead 206 may be a single pass lead having a plurality electrodes for stimulating multiple cardiac sites, including electrodes disposed at or near the His bundle (e.g., the electrodes 212A-212B) and electrodes disposed in one or more of RA, RV, LA, or LV of the heart 202. In some examples, in addition to the lead 206, the lead system may include separate leads for placement in different heart chambers or sites, such as an RA lead having one or more RA electrodes to stimulate a portion of RA or to sense RA electrical activity, a RV lead having one or more RV electrodes to stimulate a portion of RV or to sense RV electrical activity, or an LV lead having one or more LV electrodes to stimulate a portion of LV or to sense LV activity. In some examples, the cardiac disease management system 200 may include one or more leadless stimulators/sensors untethered to a lead and in wireless communication with the IMD 102. The leadless stimulators/sensors may deliver electrostimulation, sense a physiological signal, such as cardiac electrical signals in response to cardiac stimulation, and transmit the sensed data to the IMD 102.

The IMD 102 may include a hermetically sealed housing 216 that houses one or more of an electrostimulation circuit, a sensing circuit, a control circuit, a communication circuit, and a battery, among other components. In an example, the IMD 102 includes a bundle branch block (BBB) detector circuit 218 configured to detect BBB using a physiological signal sensed from the patient via one or more electrodes associated with the lead system or via a physiological sensor. Examples of the physiological signal may include an electrocardiogram (ECG), an intracardiac electrogram (EGM) such as an atrial EGM, a ventricular EGM, or a His bundle EGM, a heart rate or a pulse rate signal, a thoracic impedance signal, a cardiac impedance signal, an arterial pressure signal, a pulmonary artery pressure signal, a left atrial pressure signal, an RV pressure signal, an LV coronary pressure signal, a coronary blood temperature signal, a blood oxygen saturation signal, a cardiac acceleration signal, a heart sound signal, an intracardiac acceleration signal, a respiration signal, or a physical activity or exertion level signal, a cardiac timing signal, among others.

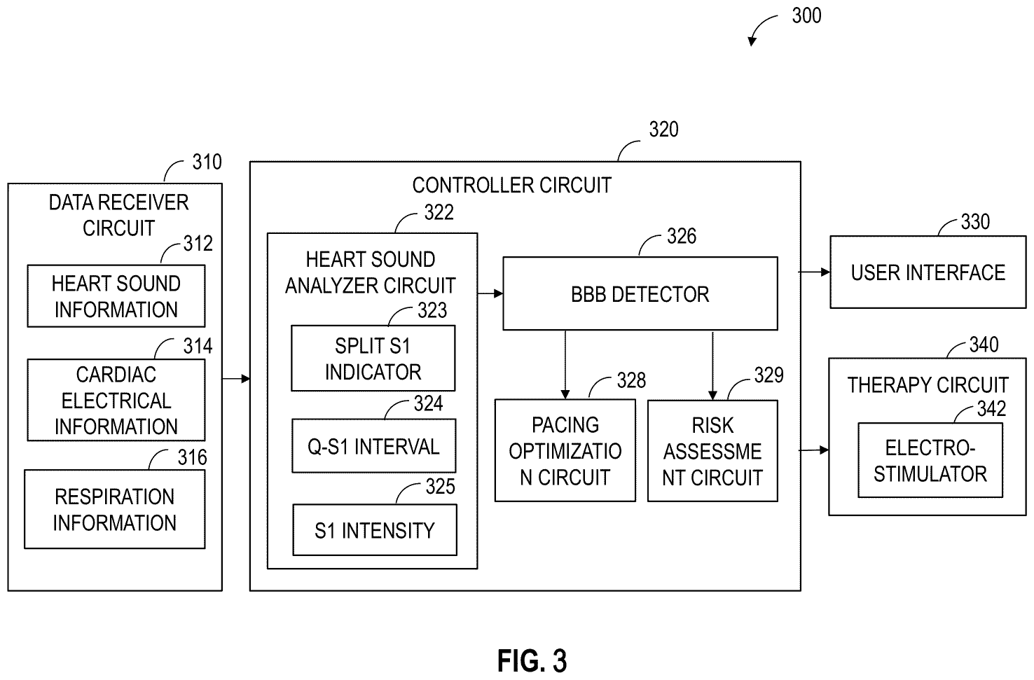

In an example, the IMD 102 may include or be coupled to a heart sound sensor to sense heart sound information. The heart sound information may include the patient's intrinsic heart sounds (e.g., in the absence of cardiac stimulation), or heart sounds sensed in response to cardiac stimulation (e.g., CSP at or near a location of the cardiac conduction system, such as His bundle pacing, or septal pacing such as left bundle branch pacing or right bundle branch pacing). The BBB detector circuit 218 may detect BBB based at least in part on a heart sound metric generated from the heart sound information. The detection of BBB includes discriminating LBBB from RBBB. Examples of the heart sound-based BBB detection and discrimination between LBBB and RBBB are described below with reference to FIG. 3.

The IMD 102 may include pacing circuitry 219 configured to provide cardiac pacing to restore cardiac synchrony or to improve cardiac performance. In an example, cardiac pacing pulses may be delivered to a right ventricular location, such as RV apex, via the RV lead 207 and the associated electrodes such as the distal electrode 231 and/or the proximal electrode 232. In an example, cardiac pacing pulses may be delivered to a location at or near the patient's cardiac conduction system, such as the His bundle 221, the bundle branches 222 or 223, the Purkinje fibers 224, or other conductive tissue of the cardiac conduction system via the lead 206 and the associated electrodes 212A or 212B. The pacing circuitry 219 may be programmed to deliver unipolar His bundle pacing, where the pacing pulses can be applied between one of the electrodes 212A-212B (e.g., as a cathode) and the housing 216 (e.g., as an anode). Alternatively, the pacing circuitry 219 may be programmed to deliver bipolar His bundle pacing, where the pacing pulse can be applied between two electrodes positioned at or near the His bundle, such as between the electrodes 212A and 212B. In some examples, electrodes used for unipolar or multipolar (e.g., bipolar or quadripolar) His bundle pacing may be selected by a system user from a plurality of candidate electrodes from a given lead or multiple separate leads comprising the pacing system, and programmed into the pacing circuitry 219. In some examples, cardiac pacing pulses may be provided by a leadless device, such as a leadless cardiac pacemakers (LCP). One or more electrodes may be distributed on the body of the LCP and in contact with His bundle region to deliver pacing pulses thereto.

The pacing circuitry 219 can be coupled to the bundle branch block (BBB) detector circuit 218, identify proper pacing locations, and determine or adjust values for one or more pacing parameters based at least on the detected BBB (e.g., LBBB or RBBB). In an example, the pacing circuitry 219 may select between a CSP (e.g., His bundle pacing or bundle branch pacing) and a right ventricular (RV) pacing at RV apex based on whether a LBBB or an RBBB is detected. In an example, the pacing circuitry 219 may determine an optimal or desired pacing location at or near the His-Purkinje system. The desired pacing location may include a desired pacing site, and/or a desired depth of insertion of a pacing lead into a His bundle or septal region of the heart. In some examples, the pacing circuitry 219 may determine a desired pacing dosage or timing parameter value. The desired pacing dosage or timing parameter value may be determined using heart sound information sensed in response to electrostimulation delivered at the desired pacing location. In some examples, the pacing circuitry 219 may provide ventricular backup pacing if the CSP does not capture the conductive tissue at the pacing site and fail to adequately activate the ventricles and restore cardiac synchrony.

The IMD 102 may communicate with the external system 105 via a communication link 111. The external system 105, as described above with respect to FIG. 1, may include a dedicated hardware/software system such as a programmer, a remote server-based patient management system, or alternatively a system defined predominantly by software running on a standard personal computer. The external system 105 may include a proximal external device such as a programmer device in proximity of the IMD 102. A clinician may manage the patient 101 through the IMD 102 via the communication link 111, including, among other operations, initiating or adjusting a pacing therapy, such as CSP. Additionally, the external system 105 may receive device data from the IMD 102 via the communication link 111. Examples of the device data may include real-time or stored physiological signals collected from the patient 101, physiological response to therapies delivered to the patient 101, or device operational status of the IMD 102 (e.g., battery status and lead impedance).

The external system 105 may monitor patient condition and the function of IMD 102. In various embodiments, the external system 105 may include a user interface to display received information to the user, and receive user input for operation control of the IMD 102. In an example, the external system 105 can be configured to verify pacing capture status, perform pacing threshold test to determine an HBP threshold. The capture verification and threshold testing may be executed periodically, or triggered by a specific event such as a user command. A user may use the external system 105 to program the IMD 102, such as to configure a pacing vector (e.g., specifying anode and cathode electrodes) to deliver HBP, or to configure a sense vector to sense a physiological signal.

In the example as shown in FIG. 2, the pacing circuitry 219 is completely within the IMD 102. In some examples, a portion or the entirety of the pacing circuitry 219 may be included in another device separate from the IMD 102, such as an external device. Examples of such external device can include the WMD 103, a portable pacing system analyzer (PSA) device, a programmer device for programming the IMD 102, among other electrostimulator devices. The external device can be coupled to one or more leads, such as the lead 206 or the lead 207, to provide electrostimulation respectively at one or more candidate pacing locations at or near the cardiac conduction system. The external device can receive heart sound information sensed from the patient in response to the electrostimulation at respective candidate locations, and determine a desired pacing location based at least on the heart sound information.

FIG. 3 illustrates generally an example of a heart sound-based cardiac monitor and pacing system 300 for monitoring cardiac condition and providing cardiac pacing to a patient. The system 300 can detect a blockage in a patient's cardiac conduction system, such as BBB (including LBBB and RBBB). Based on the detected blockage, the system 300 can initiate or adjust a pacing therapy, such as RV apical pacing or CSP. In some examples, the system 300 may determine an optimal or desired pacing location and/or pacing parameter values to more effectively capture the cardiac conductive tissue, restore synchrony, and improve the patient's cardiac performance.

The heart sound-based cardiac monitor and pacing system 300 may include one or more of a data receiver circuit 310, a controller circuit 320, a user interface 330, and a therapy circuit 340. At least a portion of the system 300 may be implemented in the IMD 102, the WMD 103, or the external system 105 such as one or more of the external device 106 or the remote device 108.

The data receiver circuit 310 may receive physiologic information from a patient. In an example, the data receiver circuit 310 may include a sense amplifier circuit configured to sense a physiologic signal from a patient via a physiologic sensor, such as an implantable, wearable, or otherwise ambulatory sensor or electrodes associated with the patient. The sensor may be incorporated into or associated with an ambulatory device such as the IMD 102 or the WMD 103. In some examples, the physiologic signals sensed from a patient may be stored in a storage device, such as an electronic medical record (EMR) system. The data receiver circuit 310 may receive the physiologic signal from the storage device, such as in response to a user command or a triggering event.

By way of example and not limitation, and as illustrated in FIG. 3, the data receiver circuit 310 may receive heart sound information 312, cardiac electrical information 314, and respiration information 316. The heart sound information 312, which can be sensed using a heart sound sensor from the patient, includes one or more of S1, S2, S3, or S4 heart sound components. In an example, the heart sound information 312 may include a body motion/vibration signal indicative of cardiac vibration, which is correlated to or indicative of heart sounds. In an example, at least one heart sound sensor can be included in the heart sound-based cardiac monitor and pacing system 300. Examples of the heart sound sensor may include an accelerometer, an acoustic sensor, a microphone, a piezo-based sensor, or other vibrational or acoustic sensors. The accelerometer can be a one-axis, a two-axis, or a three-axis accelerometer. Examples of the accelerometer may include flexible piezoelectric crystal (e.g., quartz) accelerometer or capacitive accelerometer, fabricated using micro electro-mechanical systems (MEMS) technology. The heart sound sensor may be included in the IMD 102 or the WMD 103, or disposed on a lead such as a part of the lead system associated with the IMD 102 or the WMD 103. In an example, an accelerometer (or other sensors) may sense an epicardial or endocardial acceleration (EA) signal from a portion of a heart, such as on an endocardial or epicardial surface of one of a left ventricle, a right ventricle, a left atrium, or a right atrium. The EA signal may contain components corresponding to various heart sound components such as one or more of S1, S2, S3, or S4 components.

The cardiac electrical information 314 may include, for example, surface electrocardiogram (ECG) sensed from electrodes placed on the body surface, subcutaneous ECG sensed from electrodes placed under the skin, intracardiac electrogram (EGM) sensed from the one or more implantable electrodes. The cardiac electrical information 314 may be sensed via electrodes included in or communicatively coupled to the IMD 102 or the WMD 103. The cardiac electrical information 314 may additionally include heart rate, heart rate variability, cardiac timing parameters, etc., which can be measured or determined from the cardiac electrical information 314.

The respiration information 316 may be sensed using a respiratory sensor including one of an accelerometer, a microphone, an impedance sensor, or a flow sensor. The respiration information 316 may include one or more respiration parameters. In an example, the respiration information 316 may include one or more respiratory phases within a respiratory cycle, including, for example, an inspiration phase and an expiration phase.

The heart sound information 312, cardiac electrical information 314, and respiration information 316 may be collected substantially concurrently from the same patient. In an example, the heart sound information 312, cardiac electrical information 314, and respiration information 316 may include, respectively, heart sound signal, cardiac electrical signal, and respiration signal that are time-aligned to each other. This allows accurate and reliable cross-signal measurement that may be used for BBB detection and pacing optimization, such as an Q-S1 interval between the Q wave on a ECG and the S1 heart sound on a heart sound signal within the same cardiac cycle, or a respiration-synchronized S1 heart sound corresponding to an inspiration phase of the respiration signal, as described further below.

The receiver circuit 310 may receive other physiological or functional information of the patient including, for example, physical activity signal, posture signal, a thoracic or cardiac impedance signal, arterial pressure signal, pulmonary artery pressure signal, left atrial pressure signal, RV pressure signal, LV coronary pressure signal, coronary blood temperature signal, blood oxygen saturation signal, heart sound signal, physiologic response to activity, apnea hypopnea index, one or more respiration signals such as a respiratory rate signal or a tidal volume signal, brain natriuretic peptide (BNP), blood panel, sodium and potassium levels, glucose level and other biomarkers and bio-chemical markers, among others.

The controller circuit 320 can detect blockage in a patient's cardiac conduction system such as LBBB or RBBB, optimize pacing therapy, and assess a cardiac or pulmonary risk in the patient based on the detected blockage. The controller circuit 320 can be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The controller circuit 320 may include circuit sets comprising one or more other circuits or sub-circuits, such as a heart sound analyzer circuit 322, a bundle branch block (BBB) detector 326, a pacing optimization circuit 328, and a risk assessment circuit 329. These circuits may alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The heart sound analyzer circuit 322 can preprocess the heart sound information 312, such as using a filter or a filter bank to remove or attenuate one or more of low-frequency signal baseline drift, high frequency noise, or other unwanted frequency contents. In an example, the heart sound segment may be band-pass filtered to a frequency range of approximately 5-90 Hz, or approximately 9-90 Hz. In an example, the filter may include a double or higher-order differentiator configured to calculate a double or higher-order differentiation of the heart sound signal. The heart sound analyzer circuit 322 may detect one or more heart sound components (e.g., S1, S2, S3, or S4) from the preprocessed heart sound signal. The heart sound component can be recognized based on heart sound signal amplitude, power, timing, morphology, spectrum, statistical analysis, or signal complexity analysis (e.g., signal entropy). In some examples, the heart sound analyzer circuit 322 can detect the heart sound components further using the cardiac electrical information 314, such as timings of QRS complex or R wave within a cardiac cycle, heart rates, heart rate variability, etc.

The heart sound analyzer circuit 322 can generate a heart sound metric from the received heart sound information 312. The heart sound information 312 may include the patient's intrinsic heart sounds (i.e., in the absence of cardiac stimulation). Such heart sound information is referred to as baseline heart sound information. The cardiac electrical information 314 and the respiration information 316 may similarly include, respectively, baseline cardiac electrical information and baseline respiration information collected when no cardiac stimulation is delivered to the heart. In some examples, the heart sound information 312 (optionally along with other information received by the data receiver circuit 310) may be sensed in response to cardiac stimulation. Such heart sound information is referred to as paced heart sound information. The paced heart sound information may be acquired during CSP at one or more His bundle locations or septal locations proximal to left bundle branch or right bundle branch, among other locations of the His-Purkinje system. Pacing pulses may be generated by an electrostimulator 342, which can be a part of the therapy circuit 340. In an example, the electromotor 342 can be included in the IMD 102 or the WMD 103. In another example, the electromotor 342 can be an external device separate from the IMD 102 or the WMD 103, such as being within the external device 106 (e.g., a programmer for programming the IMD 102 or the WMD 103) or a portable pacing system analyzer (PSA) device. The electrostimulator 342 can be electrically coupled to at least one electrode positionable at the two or more candidate locations to deliver the electrostimulation therein. The at least one electrode can be associated with a lead, such as the electrodes 212A and 212B associated with the lead 206 as shown in FIG. 2. The lead has a proximal end coupled to the electrostimulator 342 and a distal tip configured to be inserted into a His bundle site or a septal site (e.g., a LBBP site or a RBBP site). The distal tip can be inserted an adjustable depth into the His bundle or the septal region. Paced heart sound information may be acquired during the electrostimulation or immediately after the electrostimulation within a specific time period to account for heart sound response delay following the cessation of electrostimulation.

The heart sound analyzer circuit 322 can generate one or more heart sound metrics including, for example, a split S1 indicator 323, a Q-S1 interval 324, or an S1 intensity 325. Heart sound metrics generated from the baseline heart sound information are referred to as baseline heart sound metrics. Heart sound metrics generated from the paced heart sound information are referred to as paced heart sound metrics. The split S1 indicator 323 can indicate a change in a temporal pattern of S1, such as S1 elongation associated with a split S1 sound, which generally occurs when the mitral valve closes significantly before the tricuspid valve, allowing each valve to make separate sounds or vibrations, including a mitral valve closure component M1 and tricuspid valve closure component T1, that are detectable by a heart sound sensor. In an example, the heart sound analyzer circuit 322 can detect such temporal elongation of S1 associated with split S1 sound based on a morphology of S1 sound signal. For example, if more than one dominant peak is detected from a heart sound signal in a S1 detection window, a split S1 sound is detected. The S1 window can begin at a predetermined offset from the QRS complex or R wave of an ECG and have a predetermined window size (duration). In another example, the heart sound analyzer circuit 322 can detect elongation of S1 associated with split S1 sound based on a width of a signal envelop of the S1 sound. The signal envelop represents S1 sound signal power or energy over time. A split S1 sound is detected if the width of S1 signal envelop (e.g., measured at 75% of the S1 energy peak value) within an S1 detection window exceeds a specific signal width threshold. In yet another example, the heart sound analyzer circuit 322 can detect elongation of S1 associated with split S1 sound using a complexity measure of the S1 sound, such as a spectral entropy of S1 sound signal. The spectral entropy quantifies complexities (or non-uniformities) of power spectral magnitudes of a signal. A high spectral entropy indicates more uniformly distributed signal energy across a wide frequency range in the frequency domain, while a low spectral entropy indicates less uniformity in the signal energy distribution. A split S1 sound can be detected if the spectral entropy of S1 sound signal exceeds a spectral entropy threshold. In some examples, the heart sound analyzer circuit 322 can detect the presence or absence of split S1 sound in each cardiac cycle over a specific monitoring time period or a specific number of cardiac cycles, and calculate an occurrence rate of split S1 sound representing the number of split S1 sound detected during the monitoring period. In some examples, the heart sound analyzer circuit 322 can detect elongation of S1 associated with split S1 sound using a probabilistic model, such as a Gaussian mixture model. Data of S1 sound are assumed to be generated from a mixture of two or more Gaussian distributions with parameters that can be learned using a training set of S1 sound data set containing samples from available M1 and T1 components. The trained Gaussian Mixture Model can then be used to recognize or estimate from a test S1 sound data separate M1 and T1 components.

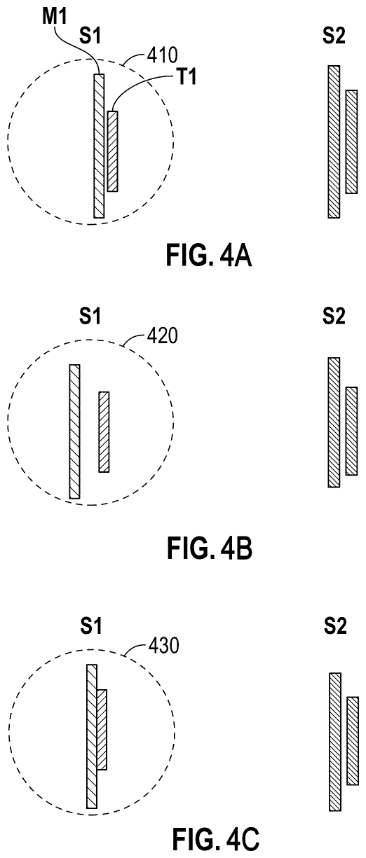

Although split S1 sound is normal in many patients, it is rarely seen in heart failure patients with LBBB. Electrostimulation, when delivered at a proper location at or near the cardiac conduction system of a heart failure patient with LBBB, can restore cardiac synchrony and promote split S1 sound pattern. Referring to FIGS. 4A-4C, the diagrams illustrate conceptually patterns of split S1 sound in normal heart (with intact cardiac conduction system) and hearts with LBBB or RBBB. S1 generally comprises a mitral valve closure component M1 and a tricuspid valve closure component T1. In a normal heart, the mitral and tricuspid valves normally close almost simultaneously, resulting in a single S1 sound. It is also normal to see slight S1 split 410 where the M1 sound occurs slightly before T1, as shown in FIG. 4A. In RBBB, the electrical impulse reaches the left ventricle before the right ventricle, causing the left ventricle to be stimulated and to contract significantly earlier than the right ventricle, allowing each valve to make a separate sound. This is presented in the heart sound signal as a wide split S1 sound 420, as shown in FIG. 4B. In LBBB, an opposite effect on S1 sound may occur due to the disruption or blockage of the left bundle branch. The electrical impulse reaches the right ventricle well before the left ventricle, such that the tricuspid valve closes earlier than the mitral valve, resulting in complete overlap of M1 and T1. This is presented in the heart sound signal as no apparent split S1 sound 430, as shown in FIG. 4C. As to be discussed further below, the BBB detector 326 can detect BBB, including to discriminate LBBB from RBBB, based on characteristics of the split S1 sound.

Referring back to FIG. 3, the Q-S1 interval 324 represents a latency between the onset of the QRS to the S1 heart sound, also known as a pre-ejection period (PEP). The heart sound analyzer circuit 322 may determine the Q-S1 interval using the heart sound information 312 and the cardiac electrical information 314 substantially concurrently collected and time-aligned with the heart sound information 312. Cardiac dyssynchrony, such as due to LBBB, can cause prolongation of Q-S1 interval. Electrostimulation at a proper location at or near the cardiac conduction system can restore cardiac synchrony and boost contractility, as reflected by a shortening of Q-S1 interval. In addition to the Q-S1 interval, the heart sound analyzer circuit 322 may determine other cardiac timing intervals that may be used for detecting BBB, optimizing pacing therapy, or assessing a cardiac or pulmonary risk. Examples of such cardiac timing interval may include a systolic timing interval (STI) representing a time interval from the onset of the QRS complex on the ECG to the S2 heart sound, a left-ventricular ejection time (LVET) representing a time interval between S1 and S2 heart sounds, or a diastolic timing interval (DTI) representing a time interval between the S2 heart sound and the onset of the subsequent QRS complex on the ECG, among others. These heart sound-based cardiac timing intervals may be correlated with cardiac contractility or cardiac diastolic function of the heart. The heart sound metrics may further include composite metrics such as PEP/LVET ratio, STI/DTI ratio, STI/cycle length (CL) ratio, or DTI/CL ratio, among others.

The S1 intensity 325 (e.g., signal amplitude or power) is associated with the vibrations produced by the heart during closure of the atrioventricular (mitral and tricuspid) valves as the ventricular pressures exceed atrial pressures at the beginning of systole. Cardiac dyssynchrony, such as due to left bundle branch block (LBBB), may cause a decrease in S1 intensity. Electrostimulation at a proper location at or near the cardiac conduction system can restore cardiac synchrony and boost contractility, as reflected by an increase in S1 intensity.

Certain respiration activities can accentuate characteristic heart sound metrics. In some examples, the heart sound analyzer circuit 322 may determine one or more heart sound metrics, such as the split S1 indicator 323, the Q-S1 interval 324, or the S1 intensity 325, using heart sound information corresponding to a particular respiration phase, which can be obtained from the respiration information 316 substantially concurrently collected and time-aligned with the heart sound information 312. For example, the split S1 indicator 323 may be determined using S1 sound corresponding to an inspiration phase in a respiration cycle. Inspiration delays the closure of the tricuspid valve due to increased venous return to right ventricle of the heart, thereby enhancing the splitting of the S1 sound and improving the BBB detection sensitivity.

The BBB detector 326 can detect BBB based at least in part on the heart sound metric generated by the heart sound analyzer circuit 322. The detection of BBB includes discriminating LBBB from RBBB. In an example, the BBB detector 326 may detect BBB based on the split S1 indicator 323. As discussed above with reference to FIGS. 4A-4C, split S1 sound is generally prominent in RBBB, but can be absent or less likely detectable in LBBB. To discriminate LBBB from RBBB, in an example, the BBB detector 326 can detect LBBB in response to an absence of the split S1 sound, or when the occurrence rate of split S1 sound is below a rate threshold. The BBB detector 326 can detect RBBB in response to a presence of the split S1 sound, or when the occurrence rate of split S1 sound is above the rate threshold. The occurrence rate can be determined during a specific monitoring time period or a specific number of cardiac cycles. The rate threshold for the split S1 sound occurrence rate can be determined using normal heart sounds of subjects with intact conduction system.

In addition or alternative to the split S1 indicator 323, the BBB detector 326 may detect BBB using one or more other heart sound metrics, such as the Q-S1 interval 324, or the S1 intensity 325. In an example, the BBB detector 326 may detect LBBB when S1 intensity 325 falls below a first threshold or falls within a first value range, and detect RBBB when S1 intensity 325 falls below a second threshold different than the first threshold, or falls within a second value range different than the first value range. In another example, the BBB detector 326 may detect LBBB when the Q-S1 interval 324 exceeds a first threshold or falls within a first value range, and detect RBBB when the Q-S1 interval 324 exceeds a second threshold different than the first threshold, or falls within a second value range different than the first value range. In an example, the BBB detector 326 may detect BBB based on variability of Q-S1 intervals measured over a specific monitoring time period or over multiple heart beats. Because of more significant and wider split S1 sound in RBBB, S1 timing (e.g., timing of S1 peak) can be more variable from beat to beat in RBBB. In contrast, in LBBB, the absence or low incidence of split S1 sound may result in narrower S1 peak and more consistent S1 timing, hence a lower beat-to-beat variability in Q-S1 timing. The BBB detector 326 can detect RBBB when the variability of Q-S1 intervals exceeds a variability threshold, and detect LBBB when the variability of Q-S1 intervals is below the variability threshold. In some examples, a primary BBB detection may be made using the split S1 indicator 323, and a secondary BBB detection based on one or more of the Q-S1 interval 324 or the S1 intensity 325 may be used to confirm the primary BBB detection. In some examples, the BBB detection (including discrimination between LBBB and RBBB) can be made using a weighted combination or a logical combination of two or more BBB detections respectively made based on the split S1 indicator 323, the Q-S1 interval 324, or the S1 intensity 325.

The pacing optimization circuit 328 can determine an optimal or desired pacing location and/or pacing parameter value based on the detection of BBB, as provided by the BBB detector 326. In an example, based on the discrimination of LBBB from RBBB, the pacing optimization circuit 328 can determine a pacing mode, such as a selection between CSP and RV pacing. In an example, when LBBB is detected, CSP can be selected to stimulate a His bundle or a septal region of the heart, such as His bundle pacing or LBB septal pacing via one or more electrodes associated with the lead 206 (shown in FIG. 2). When RBBB is detected, RV pacing can be selected to stimulate an RV apical site of the heart, such as via one or more electrodes associated with the lead 207 (shown in FIG. 2). Alternatively, in the presence of RBBB and absence of LBBB, no cardiac stimulation is delivered, so as to promote intrinsic impulses to conduct through the cardiac conduction system to the ventricles. In various examples, the pacing optimization circuit 328 can dynamically switch the pacing mode between CSP and RV pacing, depending on whether LBBB or RBBB is detected.

The pacing optimization circuit 328 can additionally or alternatively optimize lead or electrode placement for pacing therapy (e.g., CSP), such as during a procedure of lead implantation. The pacing optimization circuit 328 can determine an optimal or desired pacing location at or near the cardiac conduction system based on the discrimination of LBBB from RBBB, optionally along with one or more heart sound metrics such as the split S1 indicator 323, Q-S1 interval 324, or S1 intensity 325. In response to the detection of LBBB, CSP (e.g., His bundle pacing or LBB pacing at the septum) may be delivered to a pre-determined location at the His bundle or septal region, such as using the electrostimulator 342 of the therapy circuit 340. The electrostimulator 342 can be electrically coupled to one or more electrodes positionable at the His bundle or septal region proximal to the left bundle branch. The electrodes can be associated with a lead (e.g., the lead 207 as shown in FIG. 2), which has a distal tip configured to be inserted into the His bundle or septal region of the heart at adjustable depth of insertion to deliver the CSP therein. The data receiver circuit 310 can receive paced heart sound information from the patient in response to the CSP. The heart sound analyzer circuit 322 can detect one or more paced heart sound metrics including elongation of S1 associated with a split S1 sound using the paced heart sound information. To determine the optimal or desired pacing location (which may include an optimal or desired pacing site and an optimal or desired depth of insertion of lead tip into a His bundle or a septal region), the pacing optimization circuit 328 can determine whether or not to adjust a pacing site or a insertion depth of the distal tip of the lead based at least on the indicator of split S1 sound. As discussed above, split S1 sound pattern is usually absent and less likely detectable in LBBB. If the CSP delivered from the present pacing location produces no detectable split S1 sound pattern, or if the occurrence rate of the split S1 sound falls below a rate threshold (where the rate threshold can be a baseline occurrence rate of the split S1 sound without delivery of the CSP), then LBBB is deemed to continue in effect, and the CSP at the present location and lead tip insertion depth does not correct the cardiac dyssynchrony due to LBBB. The pacing optimization circuit 328 can generate an alert or recommendation to the user to adjust the pacing site or insertion depth, such as via the user interface 330. The recommended adjustment may include increasing the insertion depth to improve capture of the cardiac conduction system. If the CSP delivered from the present pacing location produces a detectable split S1 sound pattern, or an increase in the occurrence rate of split S1 sound that exceeds a rate threshold, then the CSP at the present location and lead tip insertion depth is deemed to correct the cardiac dyssynchrony due to LBBB. The pacing optimization circuit 328 can recommend no adjustment of the pacing site or insertion depth, and indicate to the user that the present insertion depth is acceptable for subsequent pacing therapy. In some examples, heart sound metrics such as the split S1 sound can be displayed in substantially real time on the user interface 330 to provide continuous and real-time feedback to the user as the pacing location is adjusted (e.g., as the distal tip of the lead being positioned at varying depths of insertion).

The pacing optimization circuit 328 can additionally or alternatively optimize one or more pacing parameters for a pacing therapy based on the discrimination of LBBB from RBBB and one or more heart sound metrics, such as the split S1 indicator 323, Q-S1 interval 324, or S1 intensity 325. By way of example and not limitation, the pacing therapy to be optimized can be a cardiac resynchronization pacing therapy (CRT), where both left and right ventricles of the heart are stimulated simultaneously or with a programmable interventricular delay. The pacing parameters can include, for example, a pacing dosage parameter (e.g., pulse amplitude, pulse width, pulse rate or frequency, etc.) or a pacing timing parameter. Examples of the pacing dosage parameter can include pulse amplitude, pulse width, frequency, duration(s), total charge injected per unit time, cycling (e.g., on/off time), waveform shapes, spatial locations of waveform shapes, pulse shapes, number of phases, phase order, interphase time, charge balance, among others. Examples of pacing timing parameter may include pacing delay post a sensed or paced event in another chamber, atrioventricular delay (AVD), an interventricular delay (VVD). In response to the detection of LBBB, CRT may be delivered to left and right ventricles of the heart in accordance with an initial CRT parameter (e.g., initial dosage and timing), such as using the electrostimulator 342 of the therapy circuit 340. The electrostimulator 342 can be electrically coupled to a right ventricular lead (e.g., the lead 207) and a left ventricular lead (not shown) to provided CRT therein. The data receiver circuit 310 can receive paced heart sound information sensed from the patient in response to the CRT, and the heart sound analyzer circuit 322 can detect one or more paced heart sound metrics including an elongation of S1 associated with a split S1 sound using the paced heart sound information. To determine the optimal or desired CRT parameter value, the pacing optimization circuit 328 can determine whether or not to adjust the CRT parameter based at least on the indicator of split S1 sound. If the CRT delivered in accordance with the present parameter setting produces no detectable split S1 sound pattern, or if the occurrence rate of the split S1 sound falls below a rate threshold (where the rate threshold can be a baseline occurrence rate of the split S1 sound without delivery of CRT), then LBBB is deemed to remain in effect, and the CRT at the present parameter setting does not correct the cardiac dyssynchrony due to LBBB. The pacing optimization circuit 328 can automatically adjust the CRT pacing parameter, such as by decreasing the AVD or VVD to improve cardiac synchronization between the ventricles. Alternatively, the pacing optimization circuit 328 can generate an alert or recommendation to the user to adjust the CRT pacing parameter, such as via the user interface 330. If the CRT delivered in accordance with the present parameter setting produces a detectable split S1 sound pattern, or an increase in the occurrence rate of split S1 sound that exceeds the rate threshold, then the CRT is deemed to correct the cardiac dyssynchrony due to LBBB. No adjustment of the CRT parameter will be made. A notification may be generated to indicate to the user that the present CRT parameter value is acceptable for subsequent pacing therapy. In some examples, heart sound metrics such as the split S1 sound can be displayed in substantially real time on the user interface 330 to provide continuous and real-time feedback to the user as the CRT is being delivered as the CRT parameter is adjusted.

The above techniques for optimizing CRT using heart sound metrics such as splits S1 indicator may be similarly used to optimize other cardiac pacing modalities. Conventionally, CRT involves pacing both left and right ventricles each at one pacing site, also known as single site pacing (SSP). Alternatively, multisite pacing (MSP) can be used, where electrostimulation can be delivered simultaneously or sequentially at two or more sites in a heart chamber (e.g., left ventricle) within a cardiac cycle. For example, in LV MSP, multiple LV sites may be simultaneously stimulated, or separated by one or more intra-LV time offset (ILVD). The pacing optimization circuit 328 can select between SSP and MSP based on one or more heart sound metrics, such as elongation of S1 associated with split S1 sound. For example, if the CRT produces no detectable split S1 sound pattern or if the occurrence rate of the split S1 sound falls below a rate threshold, then the pacing optimization circuit 328 can generate an alert, or recommend the user to switch from SSP to MSP, or to adjust one or more dosage or timing parameters such as ILVD. If the CRT delivered in accordance with the present parameter setting produces a detectable split S1 sound pattern, or an increase in the occurrence rate of split S1 sound that exceeds a rate threshold, then no adjustment of the CRT parameter setting is made.

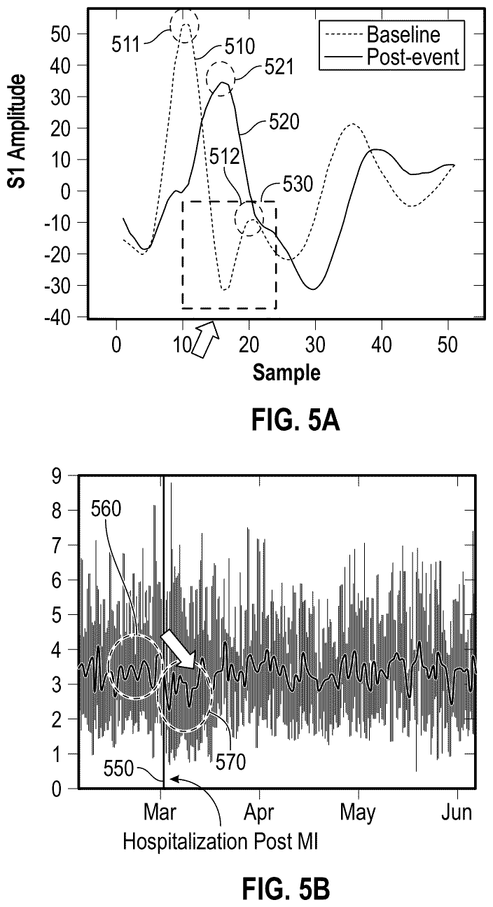

The risk assessment circuit 329 can generate a risk indicator indicating a risk of a cardiac or pulmonary event or disease progression based at least on the BBB indicator, optionally further based on one or more heart sound metrics such as the split S1 indicator 323, the Q-S1 interval 324, or the S1 intensity 325. RBBB can be resulted from pulmonary hypertension, and LBBB is a risk factor for coronary artery disease such as acute myocardial infarction (MI). Referring to FIGS. 5A-5B, the diagrams illustrate examples of changes in S1 morphology and S1 intensity before and after an MI event. FIG. 5A illustrates a segment of a baseline pre-MI S1 morphology 510 and a segment of post-MI S1 morphology 520 within a cardiac cycle, each collected from the same patient. The S1 morphologies 510 and 520 are time-aligned with respect respective R waves on respective ECG signals. The baseline S1 morphology 510 has a clear split S1 sound pattern, represented by an M1 peak 511 and a T1 peak 512 well separated in time by a trough as shown in the box 530. In the post-MI S1 morphology 520, there is no discernable split S1 sound but only one S1 peak 521. The S1 amplitude is also lower in post-MI S1 morphology 520 than in baseline pre-MI S1 morphology 510. FIG. 5B illustrates a time series of S1 intensity trended over time before and after an MI event 550. The S1 intensity is calculated as signal power (e.g., RMS value) of S1 sound. The post-MI portion 570 of the S1 intensity has a lower intensity than the pre-MI portion 560 of the S1 intensity.

Referring back to FIG. 3, if the BBB detector 326 detects the presence of LBBB, the risk assessment circuit 329 may generate a coronary artery disease risk indicative of a risk of myocardial infarction (MI) or ischemic stroke. If the BBB detector 326 detects the presence of RBBB, the risk assessment circuit 329 may generate a pulmonary hypertension risk. The cardiac or pulmonary risk can take categorical or numerical values. In an example, the cardiac or pulmonary risk be proportional to a confidence for the detected LBBB or RBBB, which can be determined based on a degree of splitting of S1 (e.g., an occurrence rate of split S1 sound), an amount of prolongation of Q-S1 interval, or an amount of decrease in S1 intensity.

The user interface 330 may include an input unit and an output unit. In an example, at least a portion of the user interface 330 may be implemented in the external system 105. The input unit may receive user input for programming the data receiver circuit 310 and the controller circuit 320, such as parameters for sensing the heart sound signal, detecting a heart sound component, computing a heart sound metric, etc. The input unit may include a keyboard, on-screen keyboard, mouse, trackball, touchpad, touch-screen, or other pointing or navigating devices. The output unit may include a display that can be configured to display information (e.g., signals) received from the data receiver circuit 310 including, among other things, heart sounds information 312, heart sound metrics generated by the heart sound analyzer circuit 322, results of BBB detection and discrimination between LBBB and RBBB generated by the BBB detector. In an example, the controller circuit 320 can generate a control signal to the user interface 330 to display in substantially real-time the heart sound metrics such as elongation of S1 associated with split S1 sound while the cardiac pacing (e.g., CSP or CRT) is being delivered as the pacing locations and/or pacing parameters are being adjusted. Optimal or desired pacing locations and/or pacing parameters determined by the pacing optimization circuit 328 can be displayed on the user interface 330 to guide lead placement and therapy programming during device and lead implantation. The cardiac or pulmonary risk generated by the risk assessment circuit 329 can be displayed on the user interface 330 or otherwise alerted to the user (e.g., clinician) to help prevent cardiac or pulmonary events (e.g., prevent acute MI, ischemic stroke, or pulmonary hypertension).

The output unit of the user interface 330 may also present to a user a recommended therapy, including a change of parameters in the therapy provided by an implanted device, the prescription to get a device implanted, the initiation or change in a drug therapy, or other treatment options of a patient. The output unit may include a printer for printing hard copies of information that may be displayed on a display unit. The signals and information may be presented in a table, a chart, a diagram, or any other types of textual, tabular, or graphical presentation formats. The presentation of the output information may include audio or other media format. In an example, the output unit may generate alerts, alarms, emergency calls, or other forms of warnings to signal the system user about the detected medical events.

The therapy circuit 340 may be configured to deliver a therapy to the patient, such as in response to the detected physiologic event. The therapy may be preventive or therapeutic in nature such as to modify, restore, or improve patient neural, cardiac, or respiratory functions. Examples of the therapy may include electrostimulation therapy delivered to the heart, a nerve tissue, other target tissues, a cardioversion therapy, a defibrillation therapy, or drug therapy including delivering drug to the patient. The therapy circuit 340 may include the electrostimulator 342 to provide cardiac pacing, such as CSP or CRT, as described above. In some examples, the therapy circuit 340 can modify an existing therapy, such as adjust a stimulation parameter or drug dosage. For example, electrostimulation may be delivered to the optimal or desired pacing location in accordance with the optimal or desired pacing dosage or timing parameter value.

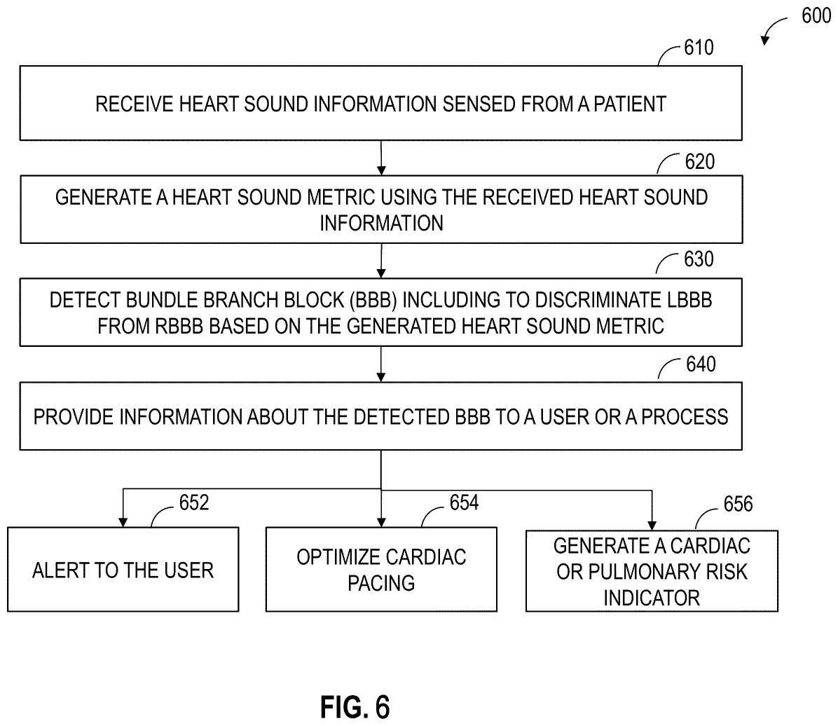

FIG. 6 illustrates generally an example method 600 for detecting and managing a blockage in a patient's cardiac conduction system such as BBB. The cardiac conduction system includes the His-Purkinje system comprising His bundle, left or right bundle branches, fascicles, or Purkinje fibers, among other conductive cardiac tissue. The method 600 may be implemented and executed in an ambulatory medical device such as an implantable or wearable medical device, or in a remote patient management system. In an example, the method 600 may be implemented in and executed by the IMD 102 or the WMD 103, the external system 105, or the heart sound-based cardiac monitor and pacing system 300.

The method 600 begins at 610 to receive heart sound information sensed from a patient. The heart sound information includes one or more of S1, S2, S3, or S4 heart sound components. The heart sounds may be detected using a sensor associated with or included in an ambulatory or wearable device, such as the IMD 102 or the WMD 103. In some examples, endocardial acceleration signals sensed from inside the heart may be used to analyze heart sounds. The heart sound information may include the patient's intrinsic heart sounds, also referred to as reference or baseline heart sound information, to distinguish from paced heart sound information that are sensed in response to electrostimulation being delivered to the heart.

At 620, a heart sound metric can be generated from at least the received heart sound information, such as using the heart sound analyzer circuit 322. The heart sound metrics can be based on one or more of the heart sound components (e.g., S1, S2, S3, or S4), optionally further based on other physiological information such as cardiac electrical information. The heart sound metrics can be correlated to the level of cardiac synchrony and indicative of cardiac performance. By way of example and not limitation, the heart sound metric may include one or more of a split S1 indicator, an S1 intensity, or a cardiac timing interval such as a pre-ejection period (PEP) or Q-S1 interval. The split S1 indicator indicates a presence or absence of elongation of S1 associated with split S1 sound, which generally occurs when the mitral valve closes significantly before the tricuspid valve, allowing each valve to make a separate sound or vibrations detectable by a heart sound sensor. Cardiac dyssynchrony, such as due to LBBB, may cause significant prolongation of Q-S1 interval and/or reduction in S1 intensity. Electrostimulation at a proper location at or near the cardiac conduction system can restore cardiac synchrony and boost contractility, as reflected by a shortening of Q-S1 interval, and/or an increase in S1 intensity.

Various techniques may be used to detect the split S1 sound. In an example, elongation of S1 associated with split S1 sound can be detected based on signal morphology of a heart sound signal. For example, if more than one dominant S1 peak is detected in a S1 detection window, a detection of split S1 is declared. In another example, the elongation of S1 associated with split S1 sound can be detected based on a width of a signal envelop of the S1 sound. The signal envelop can represent S1 sound signal power or energy over time. A split S1 is detected if the width of S1 signal envelop (e.g., measured at 75% of the S1 energy peak value) within an S1 detection window exceeds a specific signal width threshold. In yet another example, the elongation of S1 associated with split S1 sound can be detected based on a complexity measure of the S1 sound, such as a spectral entropy of S1 sound. A split S1 sound is detected if the spectral entropy of S1 sound signal exceeds a specific spectral entropy threshold. In some examples, based on the detection of presence or absence of split S1 sound, an occurrence rate of split S1 sound during a specific monitoring time period or a specific number of cardiac cycles can be determined. In some examples, the S1 split sound may be detected using the S1 sound signals corresponding to an inspiration phase. Inspiration delays the closure of the tricuspid valve due to increased venous return to right ventricle of the heart, thereby enhancing the splitting of the S1 sound and improving the BBB detection sensitivity.

At 630, bundle branch block (BBB) can be detected based at least in part on the generated heart sound metric, such as using the BBB detector 326. The detection of BBB includes discriminating LBBB from RBBB. In an example, LBBB is detected in response to an absence of split S1 sound or the occurrence rate of the split S1 sound falling below a threshold; and RBBB is detected in response to a presence of the split S1 sound or the occurrence rate of split S1 sound exceeding the threshold. The occurrence rate can be determined during a specific monitoring time period or a specific number of cardiac cycles.

In addition or alternative to the split S1 indicator 323, one or more other heart sound metrics, such as Q-S1 interval or S1 intensity, may be used detect BBB and to discriminate LBBB from RBBB. In some examples, BBB detection based on one or more of the Q-S1 interval 324 or the S1 intensity 325 may be used to confirm a primary BBB detection based on the split S1 sound. In some examples, a composite BBB detection decision can be made using a weighted combination or logical combination of two or more BBB detection decisions respectively made based on the split S1 indicator, the Q-S1 interval, or the S1 intensity.

At 640, information about the detected BBB (including the discrimination between LBBB and RBBB), can be provided to a user (e.g., a clinician), or a process such as an instance of a computer program executable in a microprocessor. The BBB detection results, including the discrimination between LBBB and RBBB, along with other information such as the received heart sounds information and heart sound metrics, may be displayed on a display screen of the user interface 330.

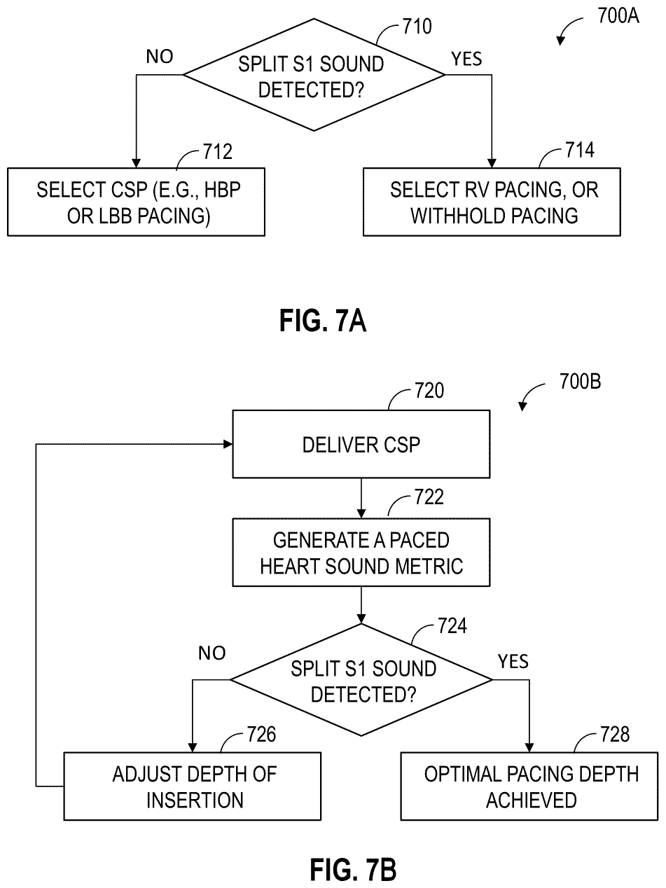

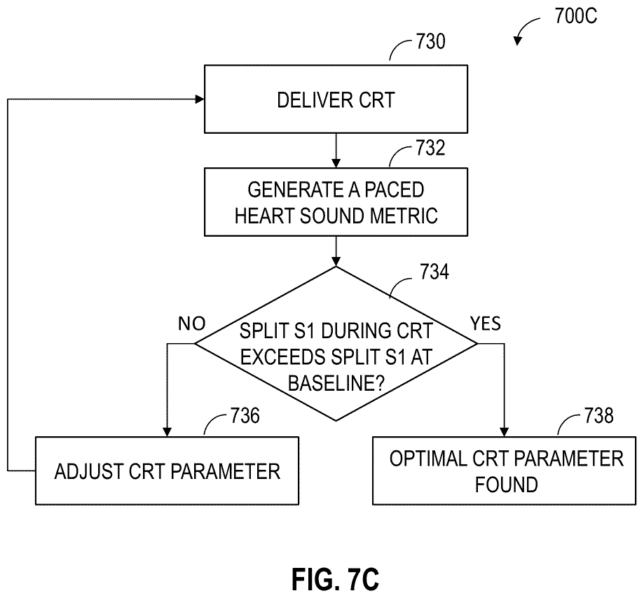

The heart sounds metrics and the detection of BBB (including the discrimination between LBBB and RBBB) can be used in several applications. At 652, an alert can be generated to inform the user about the detected BBB. Additionally or alternatively, at 654, the heart sounds metrics and the detection of BBB can be used to optimize cardiac pacing, as further discussed below with reference to FIGS. 7A-7C. In some examples, heart sound metrics (e.g., split S1 sound) can be displayed in substantially real time while cardiac pacing is being delivered as the pacing locations and/or pacing parameters are being adjusted during the process of cardiac pacing optimization. Optimal or desired pacing locations and/or pacing parameters can be displayed on the user to guide lead placement and therapy programming during device and lead implantation. Additionally or alternatively, at 656, a cardiac or pulmonary risk can be generated based at least on the detected BBB, such as using the risk assessment circuit 329. For example, a coronary artery disease risk indicative of a risk of myocardial infarction (MI) or ischemic stroke can be generated in response to a detection of LBBB, and a pulmonary hypertension risk can be generated in response to a detection of RBBB. The cardiac or pulmonary risk can take categorical or numerical values proportional to a confidence for the detected LBBB or RBBB, which can be determined based on a degree of splitting of S1 (e.g., an occurrence rate of split S1 sound), an amount of prolongation of Q-S1 interval, or an amount of decrease in S1 intensity. The cardiac or pulmonary risk can be displayed on the user interface or otherwise alerted to the user.

FIGS. 7A-7C illustrate generally example methods 700A-700C for optimizing cardiac pacing therapy using heart sound-based BBB detection to restore cardiac synchrony. The methods 700A-700C are each embodiments of cardiac pacing optimization step 654 of the method 600. The optimization of cardiac pacing therapy can be based on heart sound-based BBB detection as described above with reference to FIG. 6. The methods 700A-700C may each be implemented in and executed by portions of the heart sound-based cardiac monitor and pacing system 300, such as the pacing optimization circuit 328 and the therapy circuit 340.

FIG. 7A illustrates a method 700A for dynamically adjusting a pacing mode, such as by selecting between cardiac conduction system pacing (CSP) and right-ventricular (RV) pacing, based on the discrimination of LBBB from RBBB. As described above, LBBB can be discriminated from RBBB using elongation of S1 associated with split S1 sound. Detection of split S1 sound is performed as similarly discussed above such as at step 620 of method 600. If at 710 the split S1 is not detected, or if the occurrence rate of the split S1 sound over a monitoring period falls below a rate threshold, then an LBBB detection can be determined, and at 712 CSP can be selected for delivery to a His bundle or a septal region of the heart, such as His bundle pacing or LBB septal pacing. If at 710 the split S1 sound is detected, or if the occurrence rate of split S1 sound over a monitoring period exceeds the rate threshold, then an RBBB detection can be determined, and at 714 RV pacing can be selected for delivery to an RV apical site of the heart. Alternatively, in the presence of RBBB and absence of LBBB, no cardiac stimulation is delivered to allow natural conduction of intrinsic impulses through the cardiac conduction system.

FIG. 7B illustrates a method 700B for optimizing lead or electrode placement for a pacing therapy, such as CSP, during lead implantation in the patient. The lead or electrode placement may be optimized based on the discrimination of LBBB from RBBB and one or more heart sound metrics, such as a split S1 indicator, Q-S1 interval, or S1 intensity.

At 720, CSP (e.g., His bundle pacing or LBB septal pacing) can be delivered to a candidate location of the His bundle or septal region. The CSP can be delivered in response to the detection of LBBB using techniques as described above with respect to step 630 of method 600. The CSP can be delivered via one or more electrodes associated with a lead, such as the lead 207 as shown in FIG. 2. The lead has a distal tip configured to be inserted into the His bundle or septal region of the heart at adjustable depth of insertion to deliver the CSP therein.

At 722, one or more paced heart sound metrics can be generated from heart sound information sensed from the patient in response to the CSP, including a heart sound metric indicative of presence or absence of split S1 sound. Detection of split S1 sound can be performed using the techniques as discussed above with respect to step 620 of method 600. If at 724 the split S1 is not detected, or if the occurrence rate of the split S1 sound over a monitoring period falls below a rate threshold (where the rate threshold can be a baseline occurrence rate of the split S1 sound without delivery of the CSP), then LBBB is deemed to continue in effect, and CSP at the present location and lead tip insertion depth does not correct the cardiac dyssynchrony due to LBBB. Accordingly, at 726 the insertion depth needs to be adjusted. An alert or recommendation may be provided to the user to adjust the pacing site or insertion depth, such as increasing the insertion depth to improve capture of the cardiac conduction system.

If at 724 the split S1 is detected, or if there is an increase in the occurrence rate of split S1 sound that exceeds the rate threshold, then the CSP at the present location and lead tip insertion depth is deemed to correct the cardiac dyssynchrony due to LBBB. Accordingly, at 728 no adjustment of insertion depth is needed. A notification may be provided to the user to indicate that the present insertion depth is acceptable for subsequent pacing therapy. In some examples, heart sound metrics such as the split S1 sound can be displayed in substantially real time to provide continuous and real-time feedback to the user as the CSP is being delivered as the pacing location is adjusted (e.g., as the distal tip of the lead being positioned at varying depths of insertion).

FIG. 7C illustrates a method 700C for optimizing cardiac resynchronization pacing therapy (CRT). This may include optimizing one or more CRT parameters, such as a pacing dosage parameter (e.g., pulse amplitude, pulse width, pulse rate or frequency, etc.) or a pacing timing parameter (e.g., AVD, VVD). In certain cases, the CRT may be programmed to deliver a single site pacing (SSP) a ventricle (e.g., left ventricle) or a multisite pacing (MSP) at two or more sites of the same ventricle (e.g., left ventricle) simultaneously or separated by one or more intra-LV time offset (ILVD).

At 730, CRT may be delivered to left and right ventricles of the heart in accordance with an initial CRT parameter (e.g., initial dosage and timing). The CRT can be delivered in response to the detection of LBBB using techniques as described above with respect to step 630 of method 600. The CRT can be delivered via a right ventricular lead and a left ventricular lead. At 732, one or more paced heart sound metrics can be generated from heart sound information sensed from the patient in response to the CRT, including a heart sound metric indicative of presence or absence of split S1 sound. Detection of split S1 sound can be performed using the techniques as discussed above with respect to step 620 of method 600. If at 734 the split S1 is not detected, or if the occurrence rate of the split S1 sound over a monitoring period falls below a rate threshold (where the rate threshold can be a baseline occurrence rate of the split S1 sound without delivery of CRT), then LBBB is deemed to continue in effect, and the CSP at the present location and lead tip insertion depth does not correct the cardiac dyssynchrony due to LBBB. Accordingly, at 736 one or more CRT parameters are adjusted to improve cardiac synchronization between the ventricles. In an example, the adjustment of CRT parameter may include switching to MSP, or to modify ILVD associated with the MSP. The CRT parameter can be adjusted automatically. Alternatively, an alert or recommendation may be provided to the user to adjust the CRT parameter.

If at 734 the split S1 is detected, or if there is an increase in the occurrence rate of split S1 sound that exceeds the rate threshold, then the CRT with the present parameter setting is deemed to correct the cardiac dyssynchrony due to LBBB. Accordingly, at 738 no adjustment of CRT parameter is needed. A notification may be provided to the user to indicate that the present CRT parameter is acceptable for subsequent pacing therapy. In some examples, heart sound metrics such as the split S1 sound can be displayed in substantially real time to provide continuous and real-time feedback to the user as the CRT parameter is being adjusted.

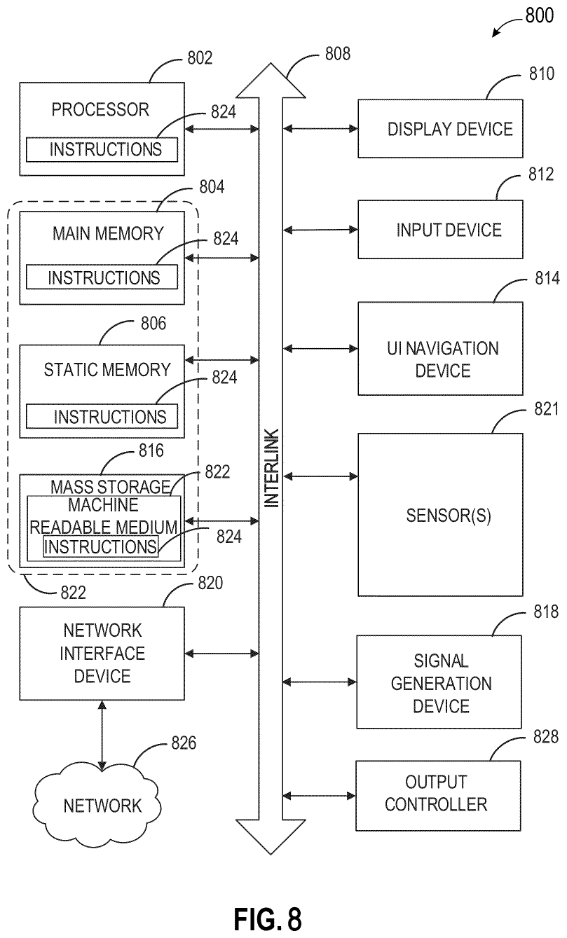

FIG. 8 illustrates generally a block diagram of an example machine 800 upon which any one or more of the techniques (e.g., methodologies) discussed herein may perform. Portions of this description may apply to the computing framework of various portions of the IMD 102, the WMD 103, the external system 105, or the heart sound-based cardiac monitor and pacing system 300.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810 (e.g., a raster display, vector display, holographic display, etc.), an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine-readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 824 may further be transmitted or received over a communication network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communication network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should therefore be determined with references to the appended claims, along with the full scope of equivalents to which such claims are entitled.

What is claimed is:

1. A medical-device system for managing a heart condition, comprising:
a data receiver circuit configured to receive heart sound information sensed from a patient, the heart sound information including an S1 sound signal; and
a controller circuit configured to:
generate a heart sound metric using the received heart sound information;
detect bundle branch block (BBB), including to discriminate a left bundle branch block (LBBB) from a right bundle branch block (RBBB), based at least in part on the generated heart sound metric; and
provide information about the detected BBB to a user or a process executable by the medical-device system.

2. The medical-device system of claim 1, wherein the heart sound metric is indicative of a presence or absence of split S1 sound,
wherein to detect the BBB, the controller circuit is configured to:
detect the LBBB in response to an absence of the split S1 sound or an occurrence rate of the split S1 sound falling below a threshold; and
detect the RBBB in response to a presence of the split S1 sound or the occurrence rate of the split S1 exceeding the threshold.

3. The medical-device system of claim 2, wherein the heart sound metric indicative of the presence or absence of split S1 sound includes a signal width of the S1 sound signal,
wherein the controller circuit is configured to detect the presence or absence of split S1 sound based on a comparison of the signal width of the S1 sound signal to a width threshold.

4. The medical-device system of claim 2, wherein the heart sound metric indicative of the presence or absence of split S1 sound includes a spectral entropy of the S1 sound signal,
wherein the controller circuit is configured to detect the presence or absence of split S1 sound based on a comparison of the spectral entropy of the S1 sound signal to a spectral entropy threshold.

5. The medical-device system of claim 2, comprising an electrostimulator configured to provide cardiac stimulation to the patient,
wherein the controller circuit is configured to generate a control signal to the electrostimulator to:
in response to the detected LBBB, deliver cardiac conduction system pacing (CSP) at a His bundle or septal region of the heart via first one or more electrodes; and
in response to the detected RBBB, withhold the cardiac stimulation, or deliver right-ventricular (RV) pacing at an RV apical site of the heart via second one or more electrodes.

6. The medical-device system of claim 5, comprising a lead having a distal portion configured to be inserted into the His bundle or septal region at adjustable depth to deliver the CSP therein via the first one or more electrodes associated with the lead,
wherein, in response to the detection of LBBB, the controller circuit is configured to:
receive paced heart sound information in response to the delivery of the CSP;
generate a heart sound metric indicative of a presence or absence of split S1 sound using the paced heart sound information; and
determine whether or not to adjust a depth of insertion of the distal portion of the lead into the His bundle or septal region, including not to adjust the depth of insertion if the heart sound metric indicates a presence of split S1 sound, or if an occurrence rate of the split S1 sound exceeds a rate threshold, and to adjust the depth of insertion if the heart sound metric indicates an absence of split S1 sound, or if the occurrence rate of the split S1 sound falls below the rate threshold.

7. The medical-device system of claim 6, wherein the controller circuit is configured to generate a control signal to a user interface to display in real time the heart sound metric indicative of the presence or absence of split S1 sound while the CSP is being delivered with the distal portion of the lead being positioned at varying depths of insertion.

8. The medical-device system of claim 5, wherein the controller circuit is configured to, in response to the detection of LBBB:
generate a control signal to the electrostimulator to deliver cardiac resynchronization pacing (CRT) to left and right ventricles of the heart in accordance with a CRT pacing parameter;
receive paced heart sound information in response to the delivery of the CRT;
generate a heart sound metric indicative of a presence or absence of split S1 sound using the paced heart sound information; and
determine whether or not to adjust the CRT pacing parameter, including not to adjust the CRT pacing parameter if the heart sound metric indicates a presence of split S1 sound, or if an occurrence rate of the split S1 sound exceeds a rate threshold, and to adjust the CRT pacing parameter if the heart sound metric indicates an absence of split S1 sound, or if the occurrence rate of the split S1 sound falls below the rate threshold.

9. The medical-device system of claim 8, wherein to adjust the CRT pacing parameter includes to adjust a pacing dosage parameter or a pacing timing parameter, or to switch from a single-site ventricular pacing to multi-site ventricular pacing.

10. The medical-device system of claim 1, wherein the data receiver circuit is configured to receive respiration information sensed from the patient, the respiration information including an inspiration phase and an expiration phase in a respiration cycle,
wherein the controller circuit is configured to generate the heart sound metric using a portion of the received heart sound information corresponding to the inspiration phase of the respiration cycle, and to detect the BBB based on the heart sound metric during the inspiration phase.

11. The medical-device system of claim 1, wherein the heart sound metric further includes one or more of an intensity of the S1 sound signal, or a pre-ejection period.

12. The medical-device system of claim 1, comprising a risk assessment circuit configured to generate a risk of cardiac or pulmonary disease in the patient based at least on the information of the detected BBB.

13. A method of managing a heart condition using a medical-device system, the method comprising:
receiving heart sound information sensed from a patient, the heart sound information including an S1 sound signal;
generating a heart sound metric using the received heart sound information;
detecting bundle branch block (BBB), including discriminating a left bundle branch block (LBBB) from a right bundle branch block (RBBB) based at least in part on the generated heart sound metric; and
providing information about the detected BBB to a user or a process.

14. The method of claim 13, wherein the heart sound metric is indicative of a presence or absence of split S1 sound,
wherein detecting the BBB includes detecting the LBBB in response to an absence of the split S1 sound or an occurrence rate of the split S1 sound falling below a threshold, and detecting the RBBB in response to a presence of the split S1 sound or the occurrence rate of the split S1 exceeding the threshold.

15. The method of claim 14, wherein the heart sound metric indicative of the presence or absence of split S1 sound includes a signal width of the S1 sound signal, the method comprising detecting the presence or absence of split S1 sound based on a comparison of the signal width of the S1 sound signal to a width threshold.

16. The method of claim 14, wherein the heart sound metric indicative of the presence or absence of split S1 sound includes a spectral entropy of the S1 sound signal, the method comprising detecting the presence or absence of split S1 sound based on a comparison of the spectral entropy of the S1 sound signal to a spectral entropy threshold.

17. The method of claim 14, comprising:
in response to the detected LBBB, delivering cardiac conduction system pacing (CSP) at a His bundle or septal region of the heart via first one or more electrodes; and
in response to the detected RBBB, withholding cardiac stimulation, or delivering right-ventricular (RV) pacing at an RV apical site of the heart via second one or more electrodes.

18. The method of claim 17, comprising, in response to the detection of LBBB:
receiving paced heart sound information in response to the delivery of the CSP at the His bundle or septal region of the heart;
generating a heart sound metric indicative of a presence or absence of split S1 sound using the paced heart sound information; and
determining whether or not to adjust a depth of insertion of a distal portion of a lead into the His bundle or septal region to deliver the CSP therein via the first one or more electrodes associated with the lead, including not adjusting the depth of insertion if the heart sound metric indicates a presence of split S1 sound, or if an occurrence rate of the split S1 sound exceeds a rate threshold, and adjusting the depth of insertion if the heart sound metric indicates an absence of split S1 sound, or if the occurrence rate of the split S1 sound falls below the rate threshold.

19. The method of claim 17, comprising, in response to the detection of LBBB:
delivering cardiac resynchronization pacing (CRT) to left and right ventricles of the heart in accordance with a CRT pacing parameter;
receiving paced heart sound information in response to the delivery of the CRT;
generating a heart sound metric indicative of a presence or absence of split S1 sound using the paced heart sound information; and
determining whether or not to adjust the CRT pacing parameter, including not adjusting the CRT pacing parameter if the heart sound metric indicates a presence of split S1 sound, or if an occurrence rate of the split S1 sound exceeds a rate threshold, and adjusting the CRT pacing parameter if the heart sound metric indicates an absence of split S1 sound, or if the occurrence rate of the split S1 sound falls below the rate threshold.

20. The method of claim 13, further comprising receiving respiration information sensed from the patient, the respiration information including an inspiration phase and an expiration phase in a respiration cycle,
wherein detecting the BBB is based at least in part on the heart sound metric that is generated using a portion of the received heart sound information corresponding to the inspiration phase of the respiration cycle.
